(19) United States
(12) Patent Application Publication
(10) Pub. No.: US 2026/0144718 A1
(43) Pub. Date: May 28, 2026

(54) SOLID POWDER COSMETIC

(71) Applicant: KOSE Corporation, Tokyo (JP)

(72) Inventor: Ryuta Gunji, Tokyo (JP)

(73) Assignee: KOSE Corporation, Tokyo (JP)

(21) Appl. No.: 19/120,937

(22) PCT Filed: Oct. 16, 2023
(86) PCT No.: PCT/JP2023/037396

§ 371 Date: Apr. 14, 2025

(30) Foreign Application Priority Data
Oct. 17, 2022 (JP) 2022-166537

(51) Int. Cl. A61K 8/02; A61Q 1/12

(52) U.S. Cl. CPC A61K 8/022; A61Q 1/12

(57) ABSTRACT

A solid powder cosmetic of the present invention provides quality that achieves a finish that feels matte and has favorable usability (the takeability of the cosmetic, the spreadability during application, and the moist feeling) and also high shock resistance. A solid powder cosmetic, comprising the following components (A) to (D):
(A) a crosslinked organopolysiloxane polymer having an alkyl branching chain and/or a phenyl branching chain on the main chain;
(B) one or more plate-like powders selected from the group consisting of mica having an average particle size of 5 to 20 μm, synthetic fluorophlogopite having an average particle size of 5 to 20 μm, and boron nitride having an average particle size of 5 to 20 μm;
(C) an oil agent that is pasty at 25° C.; and
(D) an ester oil having an IOB value of 0.15 to 0.50 and a viscosity of 10 to 10000 mPa·s at 20° C.,
wherein a content of the component (C) is 15 to 50% by mass based on the total amount of non-silicone oil agents,
a content of the component (D) is 5 to 45% by mass based on the total amount of the non-silicone oil agents, and
the cosmetic is free of a wax, or the content of the wax is 3 by mass or less based on the total amount of the cosmetic.

### TECHNICAL FIELD

The present invention relates to a solid powder cosmetic.

### BACKGROUND ART

Solid powder cosmetics are cosmetics produced in a procedure in which oily components and others are mixed with powdery components to prepare cosmetic bases, and the cosmetic bases are subjected to filling molding in containers such as a dish. Since solid powder cosmetics are easily used and carried, consumers prefer solid powder cosmetics. A matte feeling, which makes the skin look reduced in gloss and even-toned, has been desired as a texture after the application to the skin especially in recent years.

In general, the blended amount of a solidifying agent for binding powder or powders have been frequently increased to achieve a matte texture. However, if the powder is blended in a large amount, the makeup feels friction, and is more likely to feel dry. If the amounts of oily components for moisturizing the skin are increased for solving this problem, a glossy feeling is enhanced, and it may be difficult to obtain a desired matte texture. Furthermore, if the solidifying agent is blended in a large amount, the spreadability during application and the takeability (moderate adherability to an applicator or fingers) may be deteriorated. Cosmetics have been extensively examined for the compatibility between a matte finish and the usability. For example, proposed is a makeup cosmetic, comprising a silicone gel containing crosslinked polyether-modified silicone and silicone oil, an oil content, and powder, wherein the cosmetic has favorable color development properties, high makeup durability, and good takeability (for example, refer to Patent Literature 1).

Proposed are a cosmetic reduced in the dry feeling while achieving the finish of a coating film that is reduced in gloss and feels matte by combining specific amounts of two or more powders having different shapes, elastomer, a specific amount of a nonvolatile oil content, and a specific amount of wax (for example, refer to Patent Literature 2) and a solid cosmetic that is matte finished, high in spreadability during application, and obtained by combining (a) one or more powders selected from fumed silica and particulate titanium oxide, (b) a wax, (c) crosslinked organopolysiloxane, (d) a powder other than the component (a), and (e) a nonvolatile low-viscosity oil content in specific amounts (for example, refer to Patent Literature 3).

### CITATION LIST

### Patent Literature

### Patent Literature 1

Japanese Patent Laid-Open No. 2012-136495

### Patent Literature 2

Japanese Patent Laid-Open No. 2021-104936

### Patent Literature 3

Japanese Patent Laid-Open No. 2021-88517

### SUMMARY OF INVENTION

In the technique of Patent Literature 1, the cosmetic is high in takeability but feels strongly glossy, and there is room for improvement of the matte feeling. In the techniques of Patent Literatures 2 and 3, the cosmetics are excellent in finish that is reduced in gloss and feels matte and high in spreadability, but the cosmetics are hard due to the wax blended therein as solid fat, so that satisfactory takeability has not been fulfilled. Furthermore, the applied solid powder cosmetics may feel squeaky (dry), and there is room for improvement in this respect.

The present inventor has earnestly examined in view of the above-mentioned situation, and has consequently found a solid powder cosmetic comprising specific amounts of a crosslinked organopolysiloxane polymer having an alkyl branching chain or a phenyl branching chain on the main chain; one or more plate-like powders selected from mica, synthetic fluorophlogopite, and boron nitride having an average particle size of 5 to 20 μm; an oil agent that is pasty at 25° C.; and an ester oil having an IOB value of 0.15 to 0.50 and a viscosity of 10 to 10000 mPa·s at 20° C., and comprising a lower amount of a wax, while achieving a finish that feels matte, as well as favorable usability (takeability, spreadability during application, and the moist feeling of the cosmetic), leading to completion of the present invention.

That is, the present invention includes the following techniques.
[1] A solid powder cosmetic, comprising the following components (A) to (D):
(A) a crosslinked organopolysiloxane polymer having an alkyl branching chain and/or a phenyl branching chain on the main chain;
(B) one or more plate-like powders selected from the group consisting of mica having an average particle size of 5 to 20 μm, synthetic fluorophlogopite having an average particle size of 5 to 20 μm, and boron nitride having an average particle size of 5 to 20 μm;
(C) an oil agent that is pasty at 25° C.; and
(D) an ester oil having an IOB value of 0.15 to 0.50 and a viscosity of 10 to 10000 mPa·s at 20° C.,
wherein a content of the component (C) is 15 to 50% by mass based on a total amount of non-silicone oil agents,
a content of the component (D) is 5 to 45% by mass based on the total amount of the non-silicone oil agents, and
the cosmetic is free of a wax, or a content of the wax is 3% by mass or less based on a total amount of the cosmetic.
[2] The solid powder cosmetic according to [1], wherein a content mass ratio of the component (A) to a total mass of the components (C) and (D), (A)/[(C)+ (D)], is 0.25 to 2.20.
[3] The solid powder cosmetic according to [1] or [2], wherein an aspect ratio of the component (B) is 30 to 75.
[4] The solid powder cosmetic according to [1] or [2], wherein a content mass ratio of the component (B) to a total amount of the powders, (B)/(total amount of the powders), is 0.05 to 0.40.
[5] The solid powder cosmetic according to [1] or [2], wherein a content mass ratio of the powders to the total amount of the solid powder cosmetic is 0.60 to 0.85.
[6] The solid powder cosmetic according to [1] or [2], wherein the component (C) is one or more selected from the group consisting of hydrocarbon oils, dimer acid esters, sterol fatty acid esters, and dipentaerythritol fatty acid esters.
[7] The solid powder cosmetic according to [1] or [2], wherein the IOB value of the component (D) is 0.15 to 0.35.

The solid powder cosmetic of the present invention provides quality that achieves a finish that feels matte, and has favorable usability (the takeability, the spreadability during application, and the moist feeling of the cosmetic). The solid powder cosmetic of the present invention provides a quality high also in shock resistance.

### DESCRIPTION OF EMBODIMENT

Hereinafter, the present invention will be described in detail. Although the statement “% by mass” as used herein may be described merely as “%”, the statement shall mean “% by mass”. The “to” shall mean a range including the numerical values on both sides thereof. The “average particle size” described herein are measured as the median diameter by volume D50 with a laser diffraction/scattering grain size measuring apparatus (LA-910, which is manufactured by HORIBA, Ltd.). The solid powder cosmetic as used herein may be referred to merely as a “cosmetic”.

(Component (A): Crosslinked Organopolysiloxane Polymer Having Alkyl Branching Chain and/or Phenyl Branching Chain on Main Chain)

The crosslinked organopolysiloxane polymer having an alkyl branching chain and/or a phenyl branching chain on the main chain as the component (A) for the present invention is an organopolysiloxane polymer having an alkyl branching chain and/or a phenyl branching chain on the main chain (namely, it is meant that at least some of the hydrogen atoms bound to the silicon atoms of the main chain have been replaced by alkyl groups and/or phenyl groups, and the “branching chain” means a chain branching from the main chain) and exhibiting a three-dimensional structure with crosslinks. The alkyl group is preferably an alkyl group having 1 to 3 carbon atoms. The alkyl group is specifically a methyl group, an ethyl group, or a propyl group, and may be a methyl group. The crosslinked organopolysiloxane polymer having an alkyl branching chain and/or a phenyl branching chain on the main chain is a polymer obtained by crosslinking the organopolysiloxane, is also a compound that partially has three-dimensional crosslinked structure, contains a R₂SiO unit and a RSiO_{1.5} unit, and may contain a R₃SiO_{0.5} unit and/or a SiO₂ unit (wherein R of the constituent units each independently represent, for example, a group containing a hydrogen atom; an alkyl group such as a methyl group, an ethyl group, or a propyl group; an aryl group such as a phenyl group or a tolyl group; an aliphatic unsaturated group such as a vinyl group; a fluoro group; or an ethylene oxide, and the main chain has at least one alkyl group and/or at least one phenyl group).

Specific examples of the component (A) expressed as labeling names described in the “List of Labeling Name of Ingredients” made by Japan Cosmetic Industry Association include crosslinked methylpolysiloxanes such as dimethicone crosspolymer, (dimethicone/vinyl dimethicone) crosspolymer, and (vinyl dimethicone/methicone silsesquioxane) crosspolymer; and crosslinked methylphenylpolysiloxane such as (dimethicone/phenyl vinyl dimethicone) crosspolymer. Specific examples of the component (A) include the following. Examples of a polymer containing polyoxyalkylene groups in molecules thereof include crosslinked polyether-modified silicone such as (dimethicone/(PEG-10/15)) crosspolymer. Examples of a polymer containing long-chain alkyl groups in molecules thereof include crosslinked alkyl-modified silicone such as (vinyl dimethicone/lauryl dimethicone) crosspolymer. Examples of a polymer containing polyoxyalkylene groups and long-chain alkyl groups in molecules thereof include crosslinked alkyl/polyether-comodified silicone such as (PEG-15/lauryl dimethicone) crosspolymer. Examples of a polymer containing halogenated hydrocarbon groups in molecules thereof include crosslinked fluorine-modified silicone such as (trifluoropropyl dimethicone/trifluoropropyl divinyldimethicone) crosspolymer. Specific examples of the component (A) include crosslinked glycerol-modified silicone such as (dimethicone/polyglycerol-3) crosspolymer. One or more thereof can be suitably selected for use. It is preferable to use, as the component (A), especially at least one selected from the group consisting of dimethicone crosspolymer, (dimethicone/vinyl dimethicone) crosspolymer, (vinyl dimethicone/methicone silsesquioxane) crosspolymer, (vinyl dimethicone/lauryl dimethicone) crosspolymer, PEG-15/lauryl dimethicone) crosspolymer, (dimethicone/phenyl vinyl dimethicone) crosspolymer, and (dimethicone/polyglycerol-3) crosspolymer from the viewpoint of a matte feeling and spreadability during application, etc. It is more preferable to use at least one selected from the group consisting of dimethicone crosspolymer, (vinyl dimethicone/lauryl dimethicone) crosspolymer, and (PEG-15/lauryl dimethicone) crosspolymer. It is further preferable to use, as the component (A), the combination of dimethicone crosspolymer and (vinyl dimethicone/lauryl dimethicone) crosspolymer and/or the combination of dimethicone crosspolymer and (PEG-15/lauryl dimethicone) crosspolymer. In the case of the combination of dimethicone crosspolymer and (vinyl dimethicone/lauryl dimethicone) crosspolymer, dimethicone crosspolymer: (vinyl dimethicone/lauryl dimethicone) crosspolymer=1:0.05 to 1, 1:0.1 to 0.8, 1:0.1 to 0.5, or 1:0.2 to 0.5 in the content mass ratio. In case of the combination of dimethicone crosspolymer and (PEG-15/lauryl dimethicone) crosspolymer, dimethicone crosspolymer: (PEG-15/lauryl dimethicone) crosspolymer may be 1:0.05 to 1, 1:0.1 to 0.8, 1:0.1 to 0.5, or 1:0.2 to 0.5 in the content mass ratio.

The component (A) is usually powder.

The crosslinked organopolysiloxane polymer of the component (A) may be swollen with any oil agent such as a silicone oil (for example, dimethylpolysiloxane (dimethicone), methylphenylpolysiloxane (diphenylsiloxy phenyl trimethicone), or cyclopentasiloxane), an ester oil (for example, glyceryl tri-2-ethylhexanoate (triethylhexanoin)), or a hydrocarbon oil (for example, mineral oil, squalane, or isododecane). Such an oil agent for swelling is preferably one or more selected from the group consisting of dimethicone, diphenylsiloxy phenyl trimethicone, triethylhexanoin, and mineral oil; more preferably one or more selected from the group consisting of dimethicone, triethylhexanoin and mineral oil; more preferably triethylhexanoin and/or mineral oil, but not particularly limited thereto. If the crosslinked organopolysiloxane polymer is swollen state in the cosmetic, the cosmetic is high in spreadability during application, the takeability of the cosmetic and the like, and the swollen crosslinked organopolysiloxane polymer is therefore preferable. The combined use of the polymer in the swollen state and the polymer in the unswollen state is more preferable from the standpoint of the takeability of the cosmetic and the like. The content mass ratio of the unswollen polymer to the swollen polymer (solid content) may be, but not particularly limited to, 1:0.05 to 1, 1:0.1 to 0.8, or 1:0.1 to 0.5 from the viewpoint of spreadability and moist feeling. The solid content of the swollen polymer is, for example, 10 to 40%.

Examples of a commercially available mixture of the component (A) and the oil agent include KSG-15 (at a solid content of 25%) as a mixture of crosslinked methylpolysiloxane and cyclic silicone, KSG-16 (at a solid content of 20 to 30%) as a mixture of crosslinked methylpolysiloxane and dimethylpolysiloxane, KSG-18A (at a solid content of 10 to 20%) as a mixture of crosslinked methylphenylpolysiloxane and diphenylsiloxy phenyl trimethicone, KSG-210 (at a solid content of 20 to 30%) as a mixture of crosslinked polyether-modified silicone and dimethylpolysiloxane, KSG-710 (at a solid content of 20 to 30%) as a mixture of crosslinked polyglycerol-modified silicone and dimethylpolysiloxane, KSG-41 (at a solid content of 25 to 35%), KSG-42 (at a solid content of 20 to 30%), KSG-43 (at a solid content of 25 to 35%), and KSG-44 (at a solid content of 25 to 35%) as mixtures of crosslinked alkyl-modified silicone and hydrocarbon oil and/or ester oil, KSG-310 (at a solid content of 25 to 35%), KSG-320 (at a solid content of 20 to 30%), KSG-330 (at a solid content of 15 to 25%), KSG-340 (at a solid content of 25 to 35%), and KSG-340 (at a solid content of 25 to 35%) as mixtures of crosslinked alkyl/polyether-comodified silicone and hydrocarbon oil and/or ester oil (the above are manufactured by Shin-Etsu Chemical Co., Ltd.). Crosslinked fluorine-modified silicone is used as a mixture with fluorine-containing cyclic silicone such as fluoroalkyl group-containing cyclic organopolysiloxane, and examples thereof include KSG-51 (at a solid content of 15 to 25%, manufactured by Shin-Etsu Chemical Co., Ltd.).

The content of the component (A) (if the component (A) contains two or more components, the content refers to the total amount of the components as the component (A), and the same holds true for the following) for the present invention may be any content. The lower limit of the content is preferably 2.0% or more, more preferably 3.0% or more, further preferably 4.0% or more, still more preferably 4.5% or more, particularly preferably 5.0% or more of the cosmetic in terms of the solid content. The upper limit thereof is preferably 15.0% or less, more preferably 12.0% or less, further preferably 8.0% or less, still more preferably 7.5% or less, particularly preferably 7.0% or less. If the content is in this range, the cosmetic is excellent in, for example, the matte feeling, the reduced dry feeling and the like, and such a content is therefore preferable.

The content of the component (A) for the present invention is, but not particularly limited to, preferably 2.0 to 15.0%, more preferably 4.0% to 8.0%, further preferably 4.5% to 7.5%, still more preferably 5.0% to 7.0% of the cosmetic in terms of the solid content. If the content is in this range, the cosmetic is excellent in, for example, the matte feeling, the reduced dry feeling and the like, and such a content is therefore preferable.

(Component (B): One or More Plate-Like Powders Selected from Mica, Synthetic Fluorophlogopite, and Boron Nitride Having an Average Particle Size of 5 to 20 μm)

The component (B) for the present invention is plate-like powder(s) selected from mica having an average particle size of 5 to 20 μm, synthetic fluorophlogopite (synthetic mica) having an average particle size of 5 to 20 μm, and boron nitride having an average particle size of 5 to 20 μm. The term “plate-like” refers to powder particles having an aspect ratio (ratio of the major axis of the powder to the thickness thereof) of 5 or more. These can be used alone or in combination of two or more as needed (one or more types of mica, one or more types of synthetic fluorophlogopite, and one or more types of boron nitride can be naturally used in combination). Among others, the component (B) is preferably at least one selected from synthetic fluorophlogopite having an average particle size of 5 to 20 μm and boron nitride having an average particle size of 5 to 20 μm from the standpoint of spreadability during application. Since synthetic fluorophlogopite having an average particle size of 5 to 20 μm is excellent in the matte feeling, high in spreadability during application and the like, the synthetic fluorophlogopite is more preferably.

The content of the component (B) (if the component (B) contains two or more components, the content refers to the total amount of the components as the component (B), and the same holds true for the following) for the present invention may be, but not particular limited to, 3.0% or more, and is preferably 5.0% or more, more preferably 10.0% or more, further preferably 15.0% or more of the cosmetic as the lower limit. The upper limit thereof may be 35.0% or less, and is preferably 30.0% or less, more preferably 25.0% or less, further preferably 20.0% or less. If the content is in this range, the cosmetic is excellent in a matte feeling and high in spreadability during application, the takeability of the cosmetic, shock resistance and the like, and such a content is therefore preferable.

The content of the component (B) for the present invention may be, but not particularly limited to, 3.0 to 35.0% of the cosmetic, and is preferably 5.0% to 30.0%, more preferably 10.0% to 25.0%, further preferably 15.0% to 20.0% of the cosmetic. If the content is in this range, the cosmetic is excellent in a matte feeling and high in spreadability during application, the takeability of the cosmetic, shock resistance, and the like, and such a content is therefore preferable.

The average particle size of component (B) for the present invention is 5 to 20 μm. The lower limit thereof is preferably 6 μm or more, more preferably 7 μm or more, further preferably 8 μm or more. The upper limit thereof is preferably 15 μm or less, more preferably 14 μm or less, further preferably 13 μm or less. If the average particle size is in this range, the cosmetic is high in spreadability during application, excellent in the matte feeling and the like, and such an average particle size is therefore preferable. Mica, synthetic fluorophlogopite (synthetic mica), and boron nitride include raw materials having various average particle sizes from raw materials having smaller particle sizes, namely fine powders, to raw materials having larger particle sizes. In the present application, mica, synthetic fluorophlogopite (synthetic mica), and boron nitride having an average particle size that is comparatively small (but not too small) are selected and combined with the components (A), (C), and (D), so that a desired effect is exhibited.

The aspect ratio of the component (B) for the present invention is, but not particularly limited to, preferably 30 to 75, more preferably 30 to 55, still more preferably 30 to 45, particularly preferably 30 to 40. If the aspect ratio is in this range, the cosmetic is high in spreadability during application, excellent in the matte feeling and the like, and such an aspect ratio is therefore preferable.

In the present invention, the content mass ratio of the component (B) to the total amount of the powders, (B)/(total amount of powders), is, but not particularly limited to, preferably 0.05 to 0.40, more preferably 0.10 to 0.40, further preferably 0.15 to 0.35, still more preferably 0.20 to 0.30. If the ratio is in this range, the cosmetic is excellent in the matte feeling and high in spreadability during application, shock resistance and the like, and such a content mass ratio is therefore preferable. If the powder(s) is (are) surface-treated with the surface-treating agent(s), the mass(es) of the surface-treating agent(s) is (are) included in the mass(es) of the powder(s) for calculating the content mass ratio.

(Component (C): Oil Agent that is Pasty at 25° C.)

The component (C) for the present invention is an oil agent that is pasty at 25° C. The component (C) preferably has a melting point of more than 25° C. (preferably a melting point of 35 to 60° C.). A substance that is pasty at 25° C. means, for example, that the viscosity thereof at 25° C. is more than 10000 mPa·s and 100000 mPa·s or less. After a sample is left to stand at 25° C. for a day, the viscosity can be measured with a single-cylinder rotational viscometer VISMETRON type VS-A1 (manufactured by Shibaura System).

As long as the component (C) for the present invention is an oil agent for cosmetics that is pasty at 25° C., the component (C) can be used without particular limitation, but is preferably a hydrocarbon oil and/or an ester oil. Examples of the component (C) include Vaseline, pentaerythritol rosinate, (phytosteryl/isostearyl/cetyl/stearyl/behenyl)dimer dilinoleate, dipentaerythrityl hexa (hydroxystearate/stearate/rosinate), neopentyl glycol dioctanoate, phytosterol fatty acid ester, di(octyldodecyl/phytosteryl/behenyl) lauroyl glutamate, and macadamia nut fatty acid ester. Among others, the component (C) is preferably one or more selected from hydrocarbon oils, dimer acid esters, sterol fatty acid esters, and dipentaerythritol fatty acid esters; more preferably one or more selected from Vaseline, dimer acid esters, sterol fatty acid esters, and dipentaerythritol fatty acid esters.

Examples of the hydrocarbon oil include Vaseline.

Examples of the dimer acid ester include (phytosteryl/isostearyl/cetyl/stearyl/behenyl)dimer dilinoleate, dimer dilinoleyl bis(behenyl/isostearyl/phytosteryl)dimer dilinoleate, and di(isostearyl/phytosteryl)dimer dilinoleate.

Examples of the sterol fatty acid ester include di(octyl dodecyl/phytosteryl/behenyl) lauroyl glutamate, phytosteryl butyrate, phytosteryl nonanoate, cholesteryl stearate, cholesteryl isostearate, phytosteryl isostearate, cholesteryl hydroxystearate, phytosteryl hydroxystearate, phytosteryl caprylate/caprate, phytosteryl ricinoleate, cholesteryl oleate, phytosteryl oleate, dihydrocholesteryl oleate, branched fatty acid (C12-31) cholesteryl, phytosteryl canola oil fatty acid glycerides, phytosteryl rapeseed glycerides, macadamia nut oil fatty acid phytosteryl, macadamia nut oil fatty acid cholesteryl, macadamia nut oil fatty acid dihydrocholesteryl, sunflower seed oil fatty acid phytosteryl, rice bran oil fatty acid phytosteryl, and lanolin fatty acid cholesteryl.

Examples of dipentaerythritol fatty acid ester include dipentaerythritol hexaoxystearate, and dipentaerythrityl hexa (hydroxystearate/stearate/rosinate).

Among others, the component (C) is preferably at least one selected from the group consisting of Vaseline, (phytosteryl/isostearyl/cetyl/stearyl/behenyl)dimer dilinoleate, dipentaerythrityl hexa (hydroxystearate/stearate/rosinate), di(octyldodecyl/phytosteryl/behenyl) lauroyl glutamate, and macadamia nut fatty acid ester. Since the cosmetic is excellent in the spreadability during application, reduced dry feeling, shock resistance and the like, the component (C) is more preferably at least one selected from the group consisting of Vaseline, (phytosteryl/isostearyl/cetyl/stearyl/behenyl)dimer dilinoleate, and di(octyldodecyl/phytosteryl/behenyl) lauroyl glutamate. The component (C) is further preferably Vaseline from the standpoint of a matte feeling.

Examples of the commercially available component (C) for the present invention include white petrolatum (manufactured by Sonneborn LLC) as Vaseline, PLANDOOL-S (manufactured by NIPPON FINE CHEMICAL CO., LTD.) as (phytosteryl/isostearyl/cetyl/stearyl/behenyl)dimer dilinoleate, and PLANDOOL-LG1 (manufactured by NIPPON FINE CHEMICAL CO., LTD.) as di(octyldodecyl/phytosteryl/behenyl) lauroyl glutamate.

The content of the component (C) for the present invention (if the component (C) contains two or more components, the content refers to the total amount of the components as the component (C), and the same holds true for the following) may be any content. The lower limit of the content may be 1.0% or more, and is preferably 2.0% or more, more preferably 3.0% or more, further preferably 4.0% or more of the cosmetic. If the content is in this range, the cosmetic is excellent in the matte feeling and the moist feeling, high in shock resistance and the like, and such a content is therefore preferable. The upper limit thereof is preferably 8.0% or less, more preferably 7.5% or less, further preferably 7.0 or less. If the content is in this range, the cosmetic is high in spreadability during application and takeability of the cosmetic, and such a content is therefore preferable.

The content of the component (C) for the present invention is, but not particularly limited to, preferably 1.0 to 8.0%, more preferably 2.0 to 8.0%, further preferably 3.0 to 7.5%, still more preferably 4.0 to 7.0% of the cosmetic. If the content is in this range, the cosmetic is excellent in the matte feeling and the reduced dry feeling and high in shock resistance, spreadability during application, takeability of the cosmetic and the like and such a content is therefore preferable.

The content of the component (C) for the present invention based on the total amount of the non-silicone oil agents (hereinafter also referred to merely as non-silicone oil) is 15 to 50%. If the content of the component (C) based on the total amount of the non-silicone oils exceeds 50%, the spreadability during application is markedly deteriorated, and the takeability of the cosmetic is also deteriorated. If the content of the component (C) based on the total amount of the non-silicone oil is less than 15%, the moist feeling is markedly deteriorated, and the matte feeling and the shock resistance are also deteriorated. The lower limit thereof is preferably 20% or more, more preferably 25% or more, the upper limit thereof is preferably 45% or less, more preferably 40% or less, further preferably 37.5% or less, still more preferably 35% or less, and the content is preferably 20 to 40%, more preferably 25 to 37.5%, further preferably 25 to 35% from the viewpoint of shock resistance, spreadability during application and the like.

The non-silicone oil refers to an oil agent other than silicone oil agents (oil agent not having any siloxane structure in molecules thereof). Examples thereof include oil agents such as hydrocarbons, oils and fats, waxes, hardened oils, ester oils, fatty acids, higher alcohols, fluorine-containing oils, and lanolin derivatives. Specific examples include hydrocarbons such as liquid paraffin (mineral oil), squalane, Vaseline, polyisobutylene, polybutene, paraffin wax, ceresin wax, microcrystalline wax, sumac wax, montan wax, and Fischer-Tropsch wax; oils and fats such as olive oil, castor oil, mink oil, and macadamia nut oil; waxes such as beeswax, lanolin, carnauba wax, candelilla wax, and spermaceti wax; esters such as cetyl isooctanate, isopropyl myristate, isopropyl palmitate, octyldodecyl myristate, glyceryl tri-2-ethylhexanoate (triethylhexanoin), glyceryl trioctanoate, diglyceryl diisostearate, diglyceryl triisostearate, glyceryl tribehenate, pentaerythritol rosinate, (phytosteryl/isostearyl/cetyl/stearyl/behenyl)dimer dilinoleate, dipentaerythrityl hexa (hydroxystearate/stearate/rosinate), neopentyl glycol dioctanoate, cholesterol fatty acid ester, and di(cholesteryl/behenyl/octyldodecyl) N-lauroyl-L-glutamate; fatty acids such as stearic acid, lauric acid, myristic acid, behenic acid, isostearic acid, oleic acid, rosin acid, and 12-hydroxystearic acid; higher alcohols such as stearyl alcohol, cetyl alcohol, lauryl alcohol, oleyl alcohol, isostearyl alcohol, and behenyl alcohol; fluorine-containing oil agents such as perfluoropolyether, perfluorodecane, and perfluorooctene; and lanolin derivatives such as lanolin, lanolin acetate, lanolin fatty acid isopropyl, and lanolin alcohol. The non-silicone oil mentioned here includes non-silicone oil for swelling the component (A), non-silicone oil as the component (C), non-silicone oil as the component (D), and non-silicone oil as a surface-treating agent for powder.

In aspect of the present invention, the non-silicone oil is a hydrocarbon oil and/or an ester oil. An aspect of the present invention may contain a hydrocarbon oil and/or an ester oil other than the components (C) and (D) as the non-silicone oil (the hydrocarbon oil and the ester oil does not include the oil agent for swelling the component (A)), and contain a hydrocarbon oil other than the components (C) and (D). It is possible to adjust the content mass ratio of the component (C) to the total amount of non-silicone oils and the content mass ratio of the component (D) to the total amount of non-silicone oils. The content of the hydrocarbon and/or the ester oil other than the components (C) and (D) in the cosmetic is, for example, 1 to 10% by mass, or 1 to 8% by mass.

(Component (D): Ester Oil Having IOB Value of 0.15 to 0.50 and a Viscosity of 10 to 10000 mPa·s at 20° C.)

The component (D) for the present invention is an ester oil having an IOB value of 0.15 to 0.50 and a viscosity of 10 to 10000 mPa·s at 20° C. The component (D) can also be referred to as a liquid ester oil having an IOB value of 0.15 to 0.50. If the IOB value of the component (D) is less than 0.15, the cosmetic is markedly inferior in the dry feeling. If the IOB value of the component (D) exceeds 0.50, the cosmetic is markedly inferior in spreadability during application. Although detailed mechanism is unclear, it is conceivable that if the IOB value is in the above-mentioned range, the hydrophilicity is moderate, and the interaction with other components exhibits the effect of the present invention. If the viscosity of the component (D) is less than 10 mPa·s, the cosmetic is markedly inferior in spreadability during application. If the viscosity of the component (D) exceeds 10000 mPa·s, the cosmetic is markedly inferior in spreadability during application and takeability of the cosmetics.

As long as the component (D) for the present invention has an IOB value of 0.15 to 0.50 and a viscosity of 10 to 10000 mPa·s at 20° C., the component (D) may be any ester oil. Specific examples thereof include isotridecyl isononanoate (IOB=0.16, viscosity=11 mPs·s), polyglyceryl-2 tetraisostearate (IOB=0.17, viscosity=369 mPs·s), neopentyl glycol dicaprate (IOB=0.25, viscosity=19 mPs·s), diisostearyl malate (IOB=0.28, viscosity=5500 mPs·s), neopentyl glycol diethylhexanoate (IOB=0.32, viscosity=14 mPs·s), pentaerythrityl tetraethylhexanoate (IOB=0.35, viscosity=124 mPs·s), and triethylhexanoin (IOB=0.36, viscosity=30 mPs·s). One or more thereof are suitably selected for use. Among others, the component (D) is preferably at least one selected from the group consisting of polyglyceryl-2 tetraisostearate, diisostearyl malate, pentaerythrityl tetraethylhexanoate, and triethylhexanoin from the standpoint of the effect of the present invention. Since the cosmetic is high in spreadability during application, shock resistance and the like, the component (D) is more preferably at least one selected from the group consisting of polyglyceryl-2 tetraisostearate, diisostearyl malate, and pentaerythrityl tetraethylhexanoate. The component (D) is further preferably diisostearyl malate.

The IOB value is an index showing the degree of the polarity of an organic compound by the balance between the inorganicity and the organicity. The IOB value is defined as the inorganic value/the organic value. As the IOB value of a compound increases, the compound becomes more hydrophilic.

If the ester oil having an IOB value of 0.15 to 0.50 and a viscosity of 10 to 10000 mPa·s at 20° C. is used as the oil agent for swelling the component (A), the oil agent is not considered to be the component (D). If the ester oil having an IOB value of 0.15 to 0.50 and a viscosity of 10 to 10000 mPa·s at 20° C. is used as the surface-treating agent for powder, the oil agent is not considered to be the component (D), either. That is, the component (D) is an ester oil having an IOB value of 0.15 to 0.50 and a viscosity of 10 to 10000 mPa·s at 20° C. and blended as a component of the preparation. The same holds true for the component (C).

The viscosity of the component (D) for the present invention is a value measured with a Brookfield viscometer. Examples of the Brookfield viscometer include the single-cylinder rotational viscometer VISMETRON® (Shibaura System). The measuring method is as follows. A glass bottle having an outer diameter of 45 mm, an inner diameter of 38 mm, and a height of 82 mm is filled with a measurement sample without any air space, lidded, and left to stand in a thermostat at 20° C. for a day. The sample is rotated 6 to 30 times with the single-cylinder rotational viscometer VISMETRON® (Shibaura System) and rotors No. 1 to 4 attached thereto to read the measured values after 1 minute on the following day. The measured values can be multiplied by the corresponding multipliers to obtain the viscosities.

Examples of the commercially available component (D) for the present invention include COSMOL 44V (manufactured by The Nisshin OilliO Group, Ltd.) as polyglyceryl-2 tetraisostearate, COSMOL 222 (manufactured by The Nisshin OilliO Group, Ltd.) as diisostearyl malate, and SALACOS 5408 (manufactured by The Nisshin OilliO Group, Ltd.) as pentaerythrityl tetraethylhexanoate.

The content of the component (D) for the present invention (if the component (D) contains two or more components, the content refers to the total amount of the components as the component (D), and the same holds true for the following) may be, but not particularly limited to, 1.0% or more, and is preferably 1.2% or more, more preferably 1.4% or more, further preferably 1.6% or more as the lower limit, and is preferably 10.0% or less, more preferably 6.0% or less, further preferably 4.0% or less, further preferably 2.0% or less, as the upper limit. If the content is in this range, the cosmetic is high in spreadability during application and excellent in the moist feeling, the matte feeling and the like, and such a content is therefore preferable.

The content of the component (D) for the present invention based on the total amount of the cosmetic is, but not particularly limited to, preferably 1.2 to 10.0%, more preferably 1.4 to 6.0%, further preferably 1.6 to 2.0%. If the content is in this range, the cosmetic is high in spreadability during application and excellent in a moist feeling and a matte feeling, and such a content is therefore preferable.

The content of the component (D) for the present invention based on the total amount of the non-silicone oils is 5 to 45%. If the content of the component (D) based on the total amount of the non-silicone oils exceeds 45%, the matte feeling is markedly deteriorated. If the content of the component (D) based on the total amount of the non-silicone oils is less than 5%, the moist feeling is markedly deteriorated. The lower limit of the content of the component (D) based on the total amount of non-silicone oils may be 6% or more, 7% or more, 8% or more, 9% or more, or 10% or more, and the upper limit thereof is preferably 40% or less, more preferably 35% or less, further preferably 30% or less, still more preferably 25% or less, particularly preferably 20% or less, the most preferably 15% or less from the viewpoint of shock resistance and spreadability during application. The content of the component (D) based on the total amount of non-silicone oils is preferably 7 to 40%, more preferably 9 to 35%.

The upper limit of the IOB value of the component (D) for the present invention is preferably 0.45 or less, more preferably 0.40 or less, further preferably 0.35 or less. If the IOB value is in this range, the cosmetic is excellent in a moist feeling, and such an IOB value is therefore preferable. The lower limit of the IOB value of the component (D) may be 0.20 or more, or 0.25 or more. The IOB value of the component (D) for the present invention may be 0.15 to 0.45, 0.15 to 0.40, 0.20 to 0.40, or 0.25 to 0.40.

The lower limit of the viscosity of the component (D) for the present invention is preferably 15 mPa·s or more, more preferably 25 mPa·s or more, further preferably 30 mPa·s or more, and may be 50 mPa·s or more, 100 mPa·s or more, 500 mPa·s or more, or 1000 mPa·s or more. The upper limit thereof is preferably 6000 mPa·s or less, more preferably 5750 mPa·s or less, further preferably 5500 mPa·s or less. If the viscosity is in this range, the cosmetic is high in spreadability during application and the like, and such viscosity is therefore preferable.

The content mass ratio of the component (A) to the total amount of the components (C) and (D) for the present invention, (A)/[(C)+(D)], is, but not particularly limited to, preferably 0.25 to 2.20, more preferably 0.40 to 2.20, further preferably 0.55 to 2.10, still more preferably 0.70 to 2.00. If the ratio is in this range, the cosmetic is excellent in the matte feeling and high in takeability of the cosmetic and the like, and such a ratio is therefore preferable.

In the present invention, the cosmetic is free of a wax, or the content of the wax is 3% by mass or less based on the total amount of the cosmetic. Hereinafter, the wax is also referred to as a component (E). That is, the content of the wax based on the cosmetic is 0 to 3% by mass. Even when the wax is not blended, the system of the present invention guarantees the dry feeling and the shock resistance.

The component (E) in the present invention, the wax, retains the oil agent in the voids in the card house structure of the crystals thereof in the plate-like shape. The component (E) in the present invention may be any wax that is commonly used for cosmetics, and examples include synthetic hydrocarbon, natural wax, and synthetic wax. Specific examples of the component (E) include paraffin wax, ceresin wax, ozokerite wax, microcrystalline wax, Fischer Tropsch wax, polyethylene wax, ethylene-propylene copolymer, candelilla wax, carnauba wax, beeswax, rice wax, sumac wax, spermaceti wax, ozokerite, montan wax, stearyl-modified methylpolysiloxane, and behenyl-modified methylpolysiloxane. The component (E) may be limitedly paraffin wax, ceresin wax, ozokerite wax, microcrystalline wax, Fischer Tropsch wax, polyethylene wax, ethylene-propylene copolymer, candelilla wax, carnauba wax, beeswax, rice wax, sumac wax, spermaceti wax, ozokerite, montan wax, stearyl-modified methylpolysiloxane, and behenyl-modified methylpolysiloxane.

If the component (E) for the present invention is not contained, or the content thereof (if the component (E) contains two or more components, the content refers to the total amount of the two or more components) based on the total amount of the cosmetic is 3.0% or less (lower limit: 0%), the content is, but not particularly limited to, preferably 2.0% or less, more preferably 1.0% or less, further preferably 0.01% or less, and it is further preferable that the component (E) be not contained in the cosmetic. If the content is in this range, the cosmetic is high in spreadability during application, takeability of the cosmetic and the like, and such a content is therefore preferable.

A preferable aspect of the present invention is a solid powder cosmetic, comprising the following components (A) to (D):
(A) a crosslinked organopolysiloxane polymer having an alkyl branching chain and/or a phenyl branching chain on the main chain;
(B) one or more plate-like powders selected from the group consisting of mica having an average particle size of 5 to 20 μm, synthetic fluorophlogopite having an average particle size of 5 to 20 μm, and boron nitride having an average particle size of 5 to 20 μm;
(C) an oil agent that is pasty at 25° C.; and
(D) an ester oil having an IOB value of 0.15 to 0.50 and a viscosity of 10 to 10000 mPa·s at 20° C.,
   wherein the content of the component (C) is 15 to 50% by mass based on the total amount of non-silicone oil agents,
   the content of the component (D) is 5 to 45% by mass based on the total amount of the non-silicone oil agents, and
   the cosmetic is free of a wax selected from the group consisting of paraffin wax, ceresin wax, ozokerite wax, microcrystalline wax, Fischer Tropsch wax, polyethylene wax, ethylene-propylene copolymer, candelilla wax, carnauba wax, beeswax, rice wax, sumac wax, spermaceti wax, ozokerite, montan wax, stearyl-modified methylpolysiloxane, and behenyl-modified methylpolysiloxane, or the content of the wax is 3% by mass or less based on the total amount of the cosmetic.

In the solid powder cosmetic of the present invention, the content mass ratio of the powders to the total amount of the solid powder cosmetic is, but not particularly limited to, preferably 0.60 to 0.85, more preferably 0.75 to 0.82. If the content mass ratio is in this range, the cosmetic is high in shock resistance and the like, and such a content mass ratio is therefore preferable. If the powder(s) is (are) surface-treated with the surface-treating agent(s), the entire surface-treated powder(s) is (are) included for calculating the content mass ratio.

Blendable components can be optionally blended into the solid powder cosmetic in the present invention besides the components (A) to (E) as needed. For example, into the cosmetic can be blended an oil agent other than the components (C), (D), and (E), a surfactant, an alcohol, water, a humectant, a gelling agent, a thickener, powder other than the components (A) and (B), an ultraviolet absorber, an antiseptic, an antimicrobial agent, an antioxidant, skin care components (for example, a skin-whitening agent, a cell activator, an anti-inflammatory agent, a blood circulation promoter, a skin astringent, and an antiseborrheic agent), vitamins, amino acids, nucleic acids, and hormones. Since the present invention is a solid powder cosmetic, it is preferable that water be not basically contained therein. For example, the content of water may be 5% or less (lower limit: 0%), 3% or less, or 1% or less.

Powders that can be contained in the present invention are used as a coloring agent, a masking agent (makeup effect), a touch-adjusting agent, an excipient, and an ultraviolet ray-shielding agent. The powders only have to be powders for common cosmetics. The powders are not limited by shapes such as plate-like shapes and needle-like shapes; particle sizes of powders such as fumed powder, particulate powder, and pigment grade powder; and particle structures such as porous structure and non-porous structure. Examples of the powders include inorganic powders, bright powder, organic powders, dye powders, and composite powders. Specific examples include inorganic powders such as titanium oxide, black titanium oxide, Prussian blue, ultramarine blue, blood red, yellow iron oxide, black iron oxide, zinc oxide, aluminum oxide, silicic anhydride, magnesium oxide, zirconium oxide, magnesium carbonate, calcium carbonate, chromium oxide, chromium hydroxide, carbon black, aluminum silicate, magnesium silicate, aluminum magnesium silicate, mica and synthetic mica other than the component (B), sericite, talc, kaolin, silicon carbide, barium sulfate, and boron nitride other than the component (B); bright powders such as bismuth oxychloride, titanium oxide-coated mica, iron oxide-coated mica, iron oxide-coated mica titanium, organic pigment-coated mica titanium, and aluminum powder; organic powders such as wool powder, silk powder, crystalline cellulose powder, and N-acyllysine powder; dye powders such as organic tar pigment and lake pigment of organic dye; and composite powders such as particulate titanium oxide-coated mica titanium, particulate zinc oxide-coated mica titanium, barium sulfate-coated mica titanium, titanium oxide-containing silicic anhydride, and zinc oxide oxide-containing silicic anhydride. One or more of these are usable. These powders may be surface-treated with a common well-known treating agent such as a fluorine compound, a silicone compound, or a surfactant. Talc, synthetic mica, and these surface-treated powders are usable as correction materials (materials other than functional materials such as pigments imparting cosmetic effects together with the components (A) to (B)) in the solid powder cosmetic, but not particularly limited thereto. The solid powder cosmetic may contain these correction materials, for example, at 20 to 90%, or 30 to 80%.

The solid powder cosmetic of the present invention can be produced by a common well-known method. Examples include, but not particularly limited to, the following methods.

A dry molding method involving filling a container such as a metal tray or a resin tray with a cosmetic base in which the components (A) to (E) and other optional components are uniformly dispersed, followed by compression molding.

A wet molding method involving mixing a cosmetic base in which the components (A) to (E) and other optional components are uniformly dispersed with a solvent such as water, ethanol, volatile oil, or nonvolatile oil, subjecting this to filling molding, and then removing the solvent partially or entirely for molding.

Since the effect of the present invention can be particularly remarkably obtained, it is preferable to produce the solid powder cosmetic of the present invention by the wet molding method.

The solid powder cosmetic of the present invention contains the powders as the main ingredients, and is applicable to, but not particularly limited to, makeup cosmetics such as foundation, white makeup powder, eye shadow, cheek shadow, and eyebrow pencils; and basic cosmetics such as body powder and whitening powder. Among these, the solid powder cosmetic is preferably used for makeup cosmetics and further preferably used for foundation, eye shadow, and cheek shadow from the standpoint that the effect of the present invention is remarkably exhibited.

The following configurations are adoptable for the present invention.
<1> A solid powder cosmetic, comprising the following components (A) to (D):
(A) a crosslinked organopolysiloxane polymer having an alkyl branching chain and/or a phenyl branching chain on the main chain;
(B) one or more plate-like powders selected from the group consisting of mica having an average particle size of 5 to 20 μm, synthetic fluorophlogopite having an average particle size of 5 to 20 μm, and boron nitride having an average particle size of 5 to 20 μm;
(C) an oil agent that is pasty at 25° C.; and
(D) an ester oil having an IOB value of 0.15 to 0.50 and a viscosity of 10 to 10000 mPa·s at 20° C.,
   wherein a content of the component (C) is 15 to 50% by mass based on a total amount of non-silicone oil agents,
   a content of the component (D) is 5 to 45% by mass based on the total amount of the non-silicone oil agents, and
   the cosmetic is free of a wax, or a content of the wax is 3% by mass or less based on a total amount of the cosmetic.
<2> The solid powder cosmetic according to <1>, wherein a content mass ratio of the component (A) to a total mass of the components (C) and (D), (A)/[(C)+ (D)], is 0.25 to 2.20.
<3> The solid powder cosmetic according to <1> or <2>, wherein an aspect ratio of the component (B) is 30 to 75.
<4> The solid powder cosmetic according to any one of <1> to <3>, wherein a content mass ratio of the component (B) to a total amount of the powders, (B)/(total amount of the powders), is 0.05 to 0.40.
<5> The solid powder cosmetic according to any one of <1> to <4>, wherein a content mass ratio of the powders to the total amount of the solid powder cosmetic is 0.60 to 0.85.
<6> The solid powder cosmetic according to any one of <1> to <5>, wherein the component (C) is one or more selected from the group consisting of hydrocarbon oils, dimer acid esters, sterol fatty acid esters, and dipentaerythritol fatty acid esters.
<7> The solid powder cosmetic according to any one of <1> to <6>, wherein the IOB value of the component (D) is 0.15 to 0.35.

### EXAMPLES

Subsequently, the present invention will be described in further detail by giving Examples, but the present invention is not limited thereby in any way.

### Examples 1 to 33 and Comparative Examples 1 to 16: Solid Powder Cosmetics (Eye Shadows)

Solid powder cosmetics shown in Tables 1 to 5 (Tables 1-1 to 5-2) were prepared by the following method. The cosmetics were evaluated by the following methods for evaluating a. the matte feeling, b. the spreadability during application, c. the moist feeling, d. the takeability of the cosmetic, and e. the shock resistance. The Tables also show the results thereof together.

TABLE 1-1
% by mass

| | | Example | | | | | | | | | |
|---|---|---|---|---|---|---|---|---|---|---|---|
| No. | Component | 1 | 2 | 3 | 4 | 5 | 6 | 7 | 8 | 9 | 10 |
| 1 | (Dimethicone/vinyl dimethicone) | — | — | — | 4.7 | — | — | — | — | — | — |
| | crosspolymer: 25%, | | | | | | | | | | |
| | dimethicone: 75% *1 | | | | | | | | | | |
| 2 | (Dimethicone/phenyl vinyl | — | — | — | — | 7.8 | — | — | — | — | — |
| | dimethicone) crosspolymer: | | | | | | | | | | |
| | 15%, diphenylsiloxy phenyl | | | | | | | | | | |
| | trimethicone: 85% *2 | | | | | | | | | | |
| 3 | (Vinyl dimethicone/lauryl | 3.9 | 7.9 | — | — | — | — | — | 2 | 12 | 3.9 |
| | dimethicone) crosspolymer: | | | | | | | | | | |
| | 30%, triethylhexanoin: 70% *3 | | | | | | | | | | |
| 4 | (PEG-15/lauryl dimethicone) | — | — | — | — | — | 3.9 | — | — | — | — |
| | crosspolymer: 30%, | | | | | | | | | | |
| | mineral oil: 70% *4 | | | | | | | | | | |
| 5 | (Dimethicone/polyglycerol-3) | — | — | — | — | — | — | 4.7 | — | — | — |
| | crosspolymer: 25%, | | | | | | | | | | |
| | dimethicone: 75% *5 | | | | | | | | | | |
| 6 | Mixture of silica and | — | — | — | — | — | — | — | — | — | — |
| | dimethicone: solid content 30% | | | | | | | | | | |
| 7 | Dimethicone crosspolymer *6 | 4 | — | 7.9 | 4 | 4 | 4 | 4 | 4 | 4 | — |
| 8 | (Vinyl dimethicone/methicone | — | — | — | — | — | — | — | — | — | 4 |
| | silsesquioxane) crosspolymer *7 | | | | | | | | | | |
| 9 | Silica | — | — | — | — | — | — | — | — | — | — |
| 10 | Synthetic fluorophlogopite | 18 | 18 | 18 | 18 | 18 | 18 | 18 | 18 | 18 | 18 |
| | (aspect ratio: 35, average | | | | | | | | | | |
| | particle size: 12 μm) *9 | | | | | | | | | | |
| 11 | Synthetic fluorophlogopite | — | — | — | — | — | — | — | — | — | — |
| | (aspect ratio: 55, average | | | | | | | | | | |
| | particle size: 10 μm) *10 | | | | | | | | | | |
| 12 | Mica (aspect ratio: 70, | — | — | — | — | — | — | — | — | — | — |
| | average particle size: 10 μm) | | | | | | | | | | |
| | *11 | | | | | | | | | | |
| 13 | Boron nitride (aspect ratio: | — | — | — | — | — | — | — | — | — | — |
| | 31, average particle | | | | | | | | | | |
| | size: 15 μm) *12 | | | | | | | | | | |
| 14 | Synthetic fluorophlogopite | — | — | — | — | — | — | — | — | — | — |
| | (aspect ratio: 45, average | | | | | | | | | | |
| | particle size: 8 μm) *13 | | | | | | | | | | |
| 15 | Synthetic fluorophlogopite | — | — | — | — | — | — | — | — | — | — |
| | (aspect ratio: 75, average | | | | | | | | | | |
| | particle size: 20 μm) *14 | | | | | | | | | | |
| 16 | Synthetic fluorophlogopite | — | — | — | — | — | — | — | — | — | — |
| | (aspect ratio: 81, average | | | | | | | | | | |
| | particle size: 40 μm) *15 | | | | | | | | | | |
| 17 | Vaseline *16 | 6.5 | 6.5 | 6.5 | 6.5 | 6.5 | 6.5 | 6.5 | 6.5 | 6.5 | 6.5 |
| 18 | Dipentaerythrityl | — | — | — | — | — | — | — | — | — | — |
| | hexa (hydroxystearate/ | | | | | | | | | | |
| | stearate/rosinate) *17 | | | | | | | | | | |
| 19 | (Phytosteryl/isostearyl/ | — | — | — | — | — | — | — | — | — | — |
| | cetyl/stearyl/behenyl) | | | | | | | | | | |
| | dimer dilinoleate *18 | | | | | | | | | | |
| 20 | Di (octyldodecyl/ | — | — | — | — | — | — | — | — | — | — |
| | phytosteryl/behenyl) | | | | | | | | | | |
| | lauroyl glutamate *19 | | | | | | | | | | |
| 21 | Macadamia nut fatty acid | — | — | — | — | — | — | — | — | — | — |
| | phytosteryl *20 | | | | | | | | | | |
| 22 | Polyglyceryl-2 | — | — | — | — | — | — | — | — | — | — |
| | tetraisostearate | | | | | | | | | | |
| | (IOB: 0.17, viscosity: | | | | | | | | | | |
| | 369 mPa · s) *21 | | | | | | | | | | |
| 23 | Diisostearyl malate | 1.75 | 1.75 | 1.75 | 1.75 | 1.75 | 1.75 | 1.75 | 1.75 | 1.75 | 1.75 |
| | (IOB: 0.28, viscosity: | | | | | | | | | | |
| | 5500 mPa · s) *22 | | | | | | | | | | |
| 24 | Pentaerythrityl | — | — | — | — | — | — | — | — | — | — |
| | tetraethylhexanoate | | | | | | | | | | |
| | (IOB: 0.35, viscosity: | | | | | | | | | | |
| | 124 mPa · s) *23 | | | | | | | | | | |
| 25 | Triethylhexanoin | — | — | — | — | — | — | — | — | — | — |
| | (IOB: 0.36, viscosity: | | | | | | | | | | |
| | 30 mPa · s) *24 | | | | | | | | | | |
| 26 | Isononyl isononanoate | — | — | — | — | — | — | — | — | — | — |
| | (IOB: 0.20, viscosity: | | | | | | | | | | |
| | 7 mPa · s) | | | | | | | | | | |
| 27 | Diisopropyl adipate | — | — | — | — | — | — | — | — | — | — |
| | (IOB: 0.46, viscosity: | | | | | | | | | | |
| | 3 mPa · s) | | | | | | | | | | |

TABLE 1-2
% by mass

| | | Example | | | | | | | | | |
|---|---|---|---|---|---|---|---|---|---|---|---|
| No. | Component | 1 | 2 | 3 | 4 | 5 | 6 | 7 | 8 | 9 | 10 |
| 28 | Isostearyl neopentanoate | — | — | — | — | — | — | — | — | — | — |
| | (IOB: 0.14, viscosity: | | | | | | | | | | |
| | 10 mPa · s) | | | | | | | | | | |
| 29 | Polyglyceryl-2 isostearate | — | — | — | — | — | — | — | — | — | — |
| | (IOB: 0.81, viscosity: | | | | | | | | | | |
| | 6540 mPa · s) | | | | | | | | | | |
| 30 | Polyethylene wax *25 | — | — | — | — | — | — | — | — | — | — |
| 31 | Carnauba wax *26 | — | — | — | — | — | — | — | — | — | — |
| 32 | Microcrystalline wax *27 | — | — | — | — | — | — | — | — | — | — |
| 33 | Mineral oil | 6.8 | 6.8 | 6.8 | 6.8 | 6.8 | 6.8 | 6.8 | 6.8 | 6.8 | 6.8 |
| 34 | Dimethicone *28 | 2 | 2 | 2 | 2 | 2 | 2 | 2 | 2 | 2 | 2 |
| 35 | Talc | 54.05 | 54.05 | 54.05 | 54.05 | 54.05 | 54.05 | 54.05 | 55.95 | 45.95 | 54.05 |
| 36 | Iron oxide | 3 | 3 | 3 | 3 | 3 | 3 | 3 | 3 | 3 | 3 |

| | | | | | | | | | | |
|---|---|---|---|---|---|---|---|---|---|---|
| Content of (C) based on | 37 | 32 | 43 | 43 | 43 | 37 | 43 | 40 | 28 | 37 |
| total amount of non- | | | | | | | | | | |
| silicone oil agents (%) | | | | | | | | | | |
| Content of (D) based on | 10 | 9 | 12 | 12 | 12 | 10 | 12 | 11 | 7 | 10 |
| total amount of non- | | | | | | | | | | |
| silicone oil agents (%) | | | | | | | | | | |
| (A)/[(C) + (D)] | 0.63 | 0.29 | 0.96 | 0.63 | 0.63 | 0.63 | 0.63 | 0.56 | 0.92 | 0.63 |
| Content ratio of powders | 0.80 | 0.77 | 0.83 | 0.80 | 0.80 | 0.80 | 0.80 | 0.82 | 0.75 | 0.80 |
| based on total amount of | | | | | | | | | | |
| solid powder cosmetic | | | | | | | | | | |
| Content mass ratio of | 0.22 | 0.23 | 0.22 | 0.22 | 0.22 | 0.22 | 0.22 | 0.22 | 0.24 | 0.22 |
| (B) based on total | | | | | | | | | | |
| amount of powders | | | | | | | | | | |
| <Evaluation item/ | | | | | | | | | | |
| Results of determination> | | | | | | | | | | |
| Matte feeling | ⊚ | ⊚ | ⊚ | ⊚ | ◯ | ⊚ | ⊚ | ◯ | ⊚ | ⊚ |
| Spreadability during application | ⊚ | ⊚ | ◯ | ◯ | ◯ | ⊚ | ◯ | ⊚ | ◯ | ⊚ |
| Moist feeling | ⊚ | ⊚ | ⊚ | ⊚ | ⊚ | ⊚ | ⊚ | ◯ | ⊚ | ⊚ |
| Takeability of cosmetic | ⊚ | ◯ | ⊚ | ⊚ | ⊚ | ⊚ | ⊚ | ⊚ | ◯ | ⊚ |
| Shock resistance | ⊚ | ⊚ | ⊚ | ⊚ | ⊚ | ⊚ | ⊚ | ⊚ | ⊚ | ◯ |

TABLE 2-1
% by mass

| | | Example | | | | | | | | |
|---|---|---|---|---|---|---|---|---|---|---|
| No. | Component | 11 | 12 | 13 | 14 | 15 | 16 | 17 | 18 | 19 |
| 1 | (Dimethicone/vinyl dimethicone) | — | — | — | — | — | — | — | — | — |
| | crosspolymer: 25%, | | | | | | | | | |
| | dimethicone: 75% *1 | | | | | | | | | |
| 2 | (Dimethicone/phenyl vinyl | — | — | — | — | — | — | — | — | — |
| | dimethicone) crosspolymer: | | | | | | | | | |
| | 15%, diphenylsiloxy phenyl | | | | | | | | | |
| | trimethicone: 85% *2 | | | | | | | | | |
| 3 | (Vinyl dimethicone/lauryl | 3.9 | 3.9 | 3.9 | 3.9 | 3.9 | 3.9 | 3.9 | 3.9 | 3.9 |
| | dimethicone) crosspolymer: 30%, | | | | | | | | | |
| | triethylhexanoin: 70% *3 | | | | | | | | | |
| 4 | (PEG-15/lauryl dimethicone) | — | — | — | — | — | — | — | — | — |
| | crosspolymer: 30%, mineral | | | | | | | | | |
| | oil: 70% *4 | | | | | | | | | |
| 5 | (Dimethicone/polyglycerol-3) | — | — | — | — | — | — | — | — | — |
| | crosspolymer: 25%, | | | | | | | | | |
| | dimethicone: 75% *5 | | | | | | | | | |
| 6 | Mixture of silica and | — | — | — | — | — | — | — | — | — |
| | dimethicone: solid content 30% | | | | | | | | | |
| 7 | Dimethicone crosspolymer *6 | 10 | 2 | 4 | 4 | 4 | 4 | 4 | 4 | 4 |
| 8 | (Vinyl dimethicone/methicone | — | — | — | — | — | — | — | — | — |
| | silsesquioxane) crosspolymer | | | | | | | | | |
| | *7 | | | | | | | | | |
| 9 | Silica | — | — | — | — | — | — | — | — | — |
| 10 | Synthetic fluorophlogopite | 18 | 18 | — | — | — | — | — | 5 | 30 |
| | (aspect ratio: 35, average | | | | | | | | | |
| | particle size: 12 μm) *9 | | | | | | | | | |
| 11 | Synthetic fluorophlogopite | — | — | 18 | — | — | — | — | — | — |
| | (aspect ratio: 55, average | | | | | | | | | |
| | particle size: 10 μm) *10 | | | | | | | | | |
| 12 | Mica (aspect ratio: 70, average | — | — | — | 18 | — | — | — | — | — |
| | particle size: 10 μm) *11 | | | | | | | | | |
| 13 | Boron nitride (aspect ratio: | — | — | — | — | 18 | — | — | — | — |
| | 31, average particle size: | | | | | | | | | |
| | 15 μm) *12 | | | | | | | | | |
| 14 | Synthetic fluorophlogopite | — | — | — | — | — | 18 | — | — | — |
| | (aspect ratio: 45, average | | | | | | | | | |
| | particle size: 8 μm) *13 | | | | | | | | | |
| 15 | Synthetic fluorophlogopite | — | — | — | — | — | — | 18 | — | — |
| | (aspect ratio: 75, average | | | | | | | | | |
| | particle size: 20 μm) *14 | | | | | | | | | |
| 16 | Synthetic fluorophlogopite | — | — | — | — | — | — | — | — | — |
| | (aspect ratio: 81, average | | | | | | | | | |
| | particle size: 40 μm) *15 | | | | | | | | | |
| 17 | Vaseline *16 | 6.5 | 6.5 | 6.5 | 6.5 | 6.5 | 6.5 | 6.5 | 6.5 | 6.5 |
| 18 | Dipentaerythrityl hexa | — | — | — | — | — | — | — | — | — |
| | (hydroxystearate/stearate/ | | | | | | | | | |
| | rosinate) *17 | | | | | | | | | |
| 19 | (Phytosteryl/isostearyl/ | — | — | — | — | — | — | — | — | — |
| | cetyl/stearyl/behenyl) dimer | | | | | | | | | |
| | dilinoleate *18 | | | | | | | | | |
| 20 | Di (octyldodecyl/phytosteryl/ | — | — | — | — | — | — | — | — | — |
| | behenyl) lauroyl glutamate *19 | | | | | | | | | |
| 21 | Macadamia nut fatty acid | — | — | — | — | — | — | — | — | — |
| | phytosteryl *20 | | | | | | | | | |
| 22 | Polyglyceryl-2 tetraisostearate | — | — | — | — | — | — | — | — | — |
| | (IOB: 0.17, viscosity: 369 | | | | | | | | | |
| | mPa · s) *21 | | | | | | | | | |
| 23 | Diisostearyl malate | 1.75 | 1.75 | 1.75 | 1.75 | 1.75 | 1.75 | 1.75 | 1.75 | 1.75 |
| | (IOB: 0.28, viscosity: | | | | | | | | | |
| | 5500 mPa · s) *22 | | | | | | | | | |
| 24 | Pentaerythrityl | — | — | — | — | — | — | — | — | — |
| | tetraethylhexanoate | | | | | | | | | |
| | (IOB: 0.35, viscosity: | | | | | | | | | |
| | 124 mPa · s) *23 | | | | | | | | | |
| 25 | Triethylhexanoin | — | — | — | — | — | — | — | — | — |
| | (IOB: 0.36, viscosity: | | | | | | | | | |
| | 30 mPa · s) *24 | | | | | | | | | |
| 26 | Isononyl isononanoate | — | — | — | — | — | — | — | — | — |
| | (IOB: 0.20, viscosity: | | | | | | | | | |
| | 7 mPa · s) | | | | | | | | | |
| 27 | Diisopropyl adipate | — | — | — | — | — | — | — | — | — |
| | (IOB: 0.46, viscosity: | | | | | | | | | |
| | 3 mPa · s) | | | | | | | | | |
| 28 | Isostearyl neopentanoate | — | — | — | — | — | — | — | — | — |
| | (IOB: 0.14, viscosity: | | | | | | | | | |
| | 10 mPa · s) | | | | | | | | | |

TABLE 2-2
% by mass

| | | Example | | | | | | | | |
|---|---|---|---|---|---|---|---|---|---|---|
| No. | Component | 11 | 12 | 13 | 14 | 15 | 16 | 17 | 18 | 19 |
| 29 | Polyglyceryl-2 isostearate | — | — | — | — | — | — | — | — | — |
| | (IOB: 0.81, viscosity: | | | | | | | | | |
| | 6540 mPa · s) | | | | | | | | | |
| 30 | Polyethylene wax *25 | — | — | — | — | — | — | — | — | — |
| 31 | Carnauba wax *26 | — | — | — | — | — | — | — | — | — |
| 32 | Microcrystalline wax *27 | — | — | — | — | — | — | — | — | — |
| 33 | Mineral oil | 6.8 | 6.8 | 6.8 | 6.8 | 6.8 | 6.8 | 6.8 | 6.8 | 6.8 |
| 34 | Dimethicone *28 | 2 | 2 | 2 | 2 | 2 | 2 | 2 | 2 | 2 |
| 35 | Talc | 48.05 | 56.05 | 54.05 | 54.05 | 54.05 | 54.05 | 54.05 | 67.05 | 42.05 |
| 36 | Iron oxide | 3 | 3 | 3 | 3 | 3 | 3 | 3 | 3 | 3 |

| | | | | | | | | | |
|---|---|---|---|---|---|---|---|---|---|
| Content of (C) based on | 37 | 37 | 37 | 37 | 37 | 37 | 37 | 37 | 37 |
| total amount of non- | | | | | | | | | |
| silicone oil agents (%) | | | | | | | | | |
| Content of (D) based on | 10 | 10 | 10 | 10 | 10 | 10 | 10 | 10 | 10 |
| total amount of non- | | | | | | | | | |
| silicone oil agents (%) | | | | | | | | | |
| (A)/[(C) + (D)] | 1.35 | 0.38 | 0.63 | 0.63 | 0.63 | 0.63 | 0.63 | 0.63 | 0.63 |
| Content ratio of powders | 0.80 | 0.80 | 0.80 | 0.80 | 0.80 | 0.80 | 0.80 | 0.80 | 0.80 |
| based on total amount of | | | | | | | | | |
| solid powder cosmetic | | | | | | | | | |
| Content mass ratio of | 0.22 | 0.22 | 0.22 | 0.22 | 0.22 | 0.22 | 0.22 | 0.06 | 0.37 |
| (B) based on total | | | | | | | | | |
| amount of powders | | | | | | | | | |
| <Evaluation item/ | | | | | | | | | |
| Results of determination> | | | | | | | | | |
| Matte feeling | ⊚ | ◯ | ⊚ | ◯ | ◯ | ⊚ | ◯ | ◯ | ⊚ |
| Spreadability during application | ⊚ | ◯ | ◯ | ◯ | ⊚ | ◯ | ◯ | ◯ | ⊚ |
| Moist feeling | ⊚ | ⊚ | ⊚ | ⊚ | ⊚ | ⊚ | ⊚ | ⊚ | ⊚ |
| Takeability of cosmetic | ⊚ | ◯ | ⊚ | ⊚ | ⊚ | ⊚ | ⊚ | ⊚ | ⊚ |
| Shock resistance | ◯ | ⊚ | ⊚ | ⊚ | ⊚ | ⊚ | ⊚ | ⊚ | ◯ |

TABLE 3-1
% by mass

| | | Example | | | | | | | | | |
|---|---|---|---|---|---|---|---|---|---|---|---|
| No. | Component | 20 | 21 | 22 | 23 | 24 | 25 | 26 | 27 | 28 | 29 |
| 1 | (Dimethicone/vinyl dimethicone) | — | — | — | — | — | — | — | — | — | — |
| | crosspolymer: 25%, | | | | | | | | | | |
| | dimethicone: 75% *1 | | | | | | | | | | |
| 2 | (Dimethicone/phenyl vinyl | — | — | — | — | — | — | — | — | — | — |
| | dimethicone) crosspolymer: | | | | | | | | | | |
| | 15%, diphenylsiloxy phenyl | | | | | | | | | | |
| | trimethicone: 85% *2 | | | | | | | | | | |
| 3 | (Vinyl dimethicone/lauryl | 3.9 | 3.9 | 3.9 | 3.9 | 3.9 | 3.9 | 3.9 | 3.9 | 3.9 | 3.9 |
| | dimethicone) crosspolymer: | | | | | | | | | | |
| | 30%, triethylhexanoin: 70% *3 | | | | | | | | | | |
| 4 | (PEG-15/lauryl dimethicone) | — | — | — | — | — | — | — | — | — | — |
| | crosspolymer: 30%, mineral | | | | | | | | | | |
| | oil: 70% *4 | | | | | | | | | | |
| 5 | (Dimethicone/polyglycerol-3) | — | — | — | — | — | — | — | — | — | — |
| | crosspolymer: 25%, | | | | | | | | | | |
| | dimethicone: 75% *5 | | | | | | | | | | |
| 6 | Mixture of silica and | — | — | — | — | — | — | — | — | — | — |
| | dimethicone: solid content 30% | | | | | | | | | | |
| 7 | Dimethicone crosspolymer *6 | 4 | 4 | 4 | 4 | 4 | 4 | 4 | 4 | 4 | 4 |
| 8 | (Vinyl dimethicone/methicone | — | — | — | — | — | — | — | — | — | — |
| | silsesquioxane) crosspolymer *7 | | | | | | | | | | |
| 9 | Silica | — | — | — | — | — | — | — | — | — | — |
| 10 | Synthetic fluorophlogopite | 18 | 18 | 18 | 18 | 18 | 18 | 18 | 18 | 18 | 18 |
| | (aspect ratio: 35, average | | | | | | | | | | |
| | particle size: 12 μm) *9 | | | | | | | | | | |
| 11 | Synthetic fluorophlogopite | — | — | — | — | — | — | — | — | — | — |
| | (aspect ratio: 55, average | | | | | | | | | | |
| | particle size: 10 μm) *10 | | | | | | | | | | |
| 12 | Mica (aspect ratio: 70, | — | — | — | — | — | — | — | — | — | — |
| | average particle size: | | | | | | | | | | |
| | 10 μm) *11 | | | | | | | | | | |
| 13 | Boron nitride (aspect ratio: | — | — | — | — | — | — | — | — | — | — |
| | 31, average particle | | | | | | | | | | |
| | size: 15 μm) *12 | | | | | | | | | | |
| 14 | Synthetic fluorophlogopite | — | — | — | — | — | — | — | — | — | — |
| | (aspect ratio: 45, average | | | | | | | | | | |
| | particle size: 8 μm) *13 | | | | | | | | | | |
| 15 | Synthetic fluorophlogopite | — | — | — | — | — | — | — | — | — | — |
| | (aspect ratio: 75, average | | | | | | | | | | |
| | particle size: 20 μm) *14 | | | | | | | | | | |
| 16 | Synthetic fluorophlogopite | — | — | — | — | — | — | — | — | — | — |
| | (aspect ratio: 81, average | | | | | | | | | | |
| | particle size: 40 μm) *15 | | | | | | | | | | |
| 17 | Vaseline *16 | — | — | — | — | 2 | 8 | 6.5 | 6.5 | 6.5 | 6.5 |
| 18 | Dipentaerythrityl | 6.5 | — | — | — | — | — | — | — | — | — |
| | hexa (hydroxystearate/ | | | | | | | | | | |
| | stearate/rosinate) *17 | | | | | | | | | | |
| 19 | (Phytosteryl/isostearyl/ | — | 6.5 | — | — | — | — | — | — | — | — |
| | cetyl/stearyl/behenyl) | | | | | | | | | | |
| | dimer dilinoleate *18 | | | | | | | | | | |
| 20 | Di (octyldodecyl/ | — | — | 6.5 | — | — | — | — | — | — | — |
| | phytosteryl/behenyl) | | | | | | | | | | |
| | lauroyl glutamate *19 | | | | | | | | | | |
| 21 | Macadamia nut fatty | — | — | — | 6.5 | — | — | — | — | — | — |
| | acid phytosteryl *20 | | | | | | | | | | |
| 22 | Polyglyceryl-2 | — | — | — | — | — | — | 1.75 | — | — | — |
| | tetraisostearate | | | | | | | | | | |
| | (IOB: 0.17, viscosity: | | | | | | | | | | |
| | 369 mPa · s) *21 | | | | | | | | | | |
| 23 | Diisostearyl malate | 1.75 | 1.75 | 1.75 | 1.75 | 1.75 | 1.75 | — | — | — | 1.2 |
| | (IOB: 0.28, viscosity: | | | | | | | | | | |
| | 5500 mPa · s) *22 | | | | | | | | | | |
| 24 | Pentaerythrityl | — | — | — | — | — | — | — | 1.75 | — | — |
| | tetraethylhexanoate | | | | | | | | | | |
| | (IOB: 0.35, viscosity: | | | | | | | | | | |
| | 124 mPa · s) *23 | | | | | | | | | | |
| 25 | Triethylhexanoin | — | — | — | — | — | — | — | — | 1.75 | — |
| | (IOB: 0.36, viscosity: | | | | | | | | | | |
| | 30 mPa · s) *24 | | | | | | | | | | |
| 26 | Isononyl isononanoate | — | — | — | — | — | — | — | — | — | — |
| | (IOB: 0.20, viscosity: | | | | | | | | | | |
| | 7 mPa · s) | | | | | | | | | | |
| 27 | Diisopropyl adipate | — | — | — | — | — | — | — | — | — | — |
| | (IOB: 0.46, viscosity: | | | | | | | | | | |
| | 3 mPa · s) | | | | | | | | | | |
| 28 | Isostearyl neopentanoate | — | — | — | — | — | — | — | — | — | — |
| | (IOB: 0.14, viscosity: | | | | | | | | | | |
| | 10 mPa · s) | | | | | | | | | | |

TABLE 3-2
% by mass

| | | Example | | | | | | | | | |
|---|---|---|---|---|---|---|---|---|---|---|---|
| No. | Component | 20 | 21 | 22 | 23 | 24 | 25 | 26 | 27 | 28 | 29 |
| 29 | Polyglyceryl-2 isostearate | — | — | — | — | — | — | — | — | — | — |
| | (IOB: 0.81, viscosity: | | | | | | | | | | |
| | 6540 mPa · s) | | | | | | | | | | |
| 30 | Polyethylene wax *25 | — | — | — | — | — | — | — | — | — | — |
| 31 | Carnauba wax *26 | — | — | — | — | — | — | — | — | — | — |
| 32 | Microcrystalline wax *27 | — | — | — | — | — | — | — | — | — | — |
| 33 | Mineral oil | 6.8 | 6.8 | 6.8 | 6.8 | 6.8 | 6.8 | 6.8 | 6.8 | 6.8 | 6.8 |
| 34 | Dimethicone *28 | 2 | 2 | 2 | 2 | 2 | 2 | 2 | 2 | 2 | 2 |
| 35 | Talc | 54.05 | 54.05 | 54.05 | 54.05 | 58.55 | 52.55 | 54.05 | 54.05 | 54.05 | 54.6 |
| 36 | Iron oxide | 3 | 3 | 3 | 3 | 3 | 3 | 3 | 3 | 3 | 3 |

| | | | | | | | | | | |
|---|---|---|---|---|---|---|---|---|---|---|
| Content of (C) based on | 37 | 37 | 37 | 37 | 15 | 41 | 37 | 37 | 37 | 38 |
| total amount of non- | | | | | | | | | | |
| silicone oil agents (%) | | | | | | | | | | |
| Content of (D) based on | 10 | 10 | 10 | 10 | 13 | 9 | 10 | 10 | 10 | 7 |
| total amount of non- | | | | | | | | | | |
| silicone oil agents (%) | | | | | | | | | | |
| (A)/[(C) + (D)] | 0.63 | 0.63 | 0.63 | 0.63 | 1.38 | 0.53 | 0.63 | 0.63 | 0.63 | 0.67 |
| Content ratio of powders | 0.80 | 0.80 | 0.80 | 0.80 | 0.85 | 0.79 | 0.80 | 0.80 | 0.80 | 0.81 |
| based on total amount | | | | | | | | | | |
| of solid powder cosmetic | | | | | | | | | | |
| Content mass ratio of (B) | 0.22 | 0.22 | 0.22 | 0.22 | 0.21 | 0.23 | 0.22 | 0.22 | 0.22 | 0.22 |
| based on total amount of powders | | | | | | | | | | |
| <Evaluation item/ | | | | | | | | | | |
| Results of determination> | | | | | | | | | | |
| Matte feeling | ◯ | ◯ | ◯ | ◯ | ◯ | ⊚ | ⊚ | ⊚ | ⊚ | ⊚ |
| Spreadability | ◯ | ⊚ | ⊚ | ◯ | ⊚ | ◯ | ⊚ | ⊚ | ◯ | ◯ |
| during application | | | | | | | | | | |
| Moist feeling | ⊚ | ⊚ | ⊚ | ⊚ | ◯ | ⊚ | ◯ | ◯ | ◯ | ◯ |
| Takeability of cosmetic | ⊚ | ⊚ | ⊚ | ⊚ | ⊚ | ◯ | ⊚ | ⊚ | ⊚ | ⊚ |
| Shock resistance | ⊚ | ⊚ | ⊚ | ⊚ | ◯ | ⊚ | ⊚ | ⊚ | ◯ | ⊚ |

TABLE 4-1
% by mass

| | | Example | | | | Comparative Example | | | | | |
|---|---|---|---|---|---|---|---|---|---|---|---|
| No. | Component | 30 | 31 | 32 | 33 | 1 | 2 | 3 | 4 | 5 | 6 |
| 1 | (Dimethicone/vinyl | — | — | — | — | — | — | — | — | — | — |
| | dimethicone) crosspolymer: | | | | | | | | | | |
| | 25%, dimethicone: 75% *1 | | | | | | | | | | |
| 2 | (Dimethicone/phenyl vinyl | — | — | — | — | — | — | — | — | — | — |
| | dimethicone) crosspolymer: | | | | | | | | | | |
| | 15%, diphenylsiloxy phenyl | | | | | | | | | | |
| | trimethicone: 85% *2 | | | | | | | | | | |
| 3 | (Vinyl dimethicone/lauryl | 3.9 | 3.9 | 3.9 | 3.9 | — | 3.9 | 3.9 | 3.9 | 3.9 | 3.9 |
| | dimethicone) crosspolymer: | | | | | | | | | | |
| | 30%, triethylhexanoin: 70% *3 | | | | | | | | | | |
| 4 | (PEG-15/lauryl dimethicone) | — | — | — | — | — | — | — | — | — | — |
| | crosspolymer: 30%, mineral | | | | | | | | | | |
| | oil: 70% *4 | | | | | | | | | | |
| 5 | (dimethicone/polyglycerol-3) | — | — | — | — | — | — | — | — | — | — |
| | crosspolymer: 25%, | | | | | | | | | | |
| | dimethicone: 75% *5 | | | | | | | | | | |
| 6 | Mixture of silica and | — | — | — | — | — | — | — | — | — | — |
| | dimethicone: solid content 30% | | | | | | | | | | |
| 7 | Dimethicone crosspolymer *6 | 4 | 4 | 4 | 4 | — | 4 | 4 | 4 | 4 | 4 |
| 8 | (Vinyl dimethicone/ | — | — | — | — | — | — | — | — | — | — |
| | methicone silsesquioxane) | | | | | | | | | | |
| | crosspolymer *7 | | | | | | | | | | |
| 9 | Silica | — | — | — | — | — | — | — | — | — | — |
| 10 | Synthetic fluorophlogopite | 18 | 18 | 18 | 18 | 18 | — | 18 | 18 | 18 | 18 |
| | (aspect ratio: 35, average | | | | | | | | | | |
| | particle size: 12 μm) *9 | | | | | | | | | | |
| 11 | Synthetic fluorophlogopite | — | — | — | — | — | — | — | — | — | — |
| | (aspect ratio: 55, average | | | | | | | | | | |
| | particle size: 10 μm) *10 | | | | | | | | | | |
| 12 | Mica (aspect ratio: 70, | — | — | — | — | — | — | — | — | — | — |
| | average particle size: | | | | | | | | | | |
| | 10 μm) *11 | | | | | | | | | | |
| 13 | Boron nitride (aspect ratio: | — | — | — | — | — | — | — | — | — | — |
| | 31, average particle size: | | | | | | | | | | |
| | 15 μm) *12 | | | | | | | | | | |
| 14 | Synthetic fluorophlogopite | — | — | — | — | — | — | — | — | — | — |
| | (aspect ratio: 45, average | | | | | | | | | | |
| | particle size: 8 μm) *13 | | | | | | | | | | |
| 15 | Synthetic fluorophlogopite | — | — | — | — | — | — | — | — | — | — |
| | (aspect ratio: 75, average | | | | | | | | | | |
| | particle size: 20 μm) *14 | | | | | | | | | | |
| 16 | Synthetic fluorophlogopite | — | — | — | — | — | — | — | — | — | — |
| | (aspect ratio: 81, average | | | | | | | | | | |
| | particle size: 40 μm) *15 | | | | | | | | | | |
| 17 | Vaseline *16 | 6.5 | 6.5 | 6.5 | 6.5 | 6.5 | 6.5 | — | 6.5 | 15 | 6.5 |
| 18 | Dipentaerythrityl | — | — | — | — | — | — | — | — | — | — |
| | hexa (hydroxystearate/ | | | | | | | | | | |
| | stearate/rosinate) *17 | | | | | | | | | | |
| 19 | (Phytosteryl/isostearyl/ | — | — | — | — | — | — | — | — | — | — |
| | cetyl/stearyl/behenyl) dimer | | | | | | | | | | |
| | dilinoleate *18 | | | | | | | | | | |
| 20 | Di (octyldodecyl/ | — | — | — | — | — | — | — | — | — | — |
| | phytosteryl/behenyl) | | | | | | | | | | |
| | lauroyl glutamate *19 | | | | | | | | | | |
| 21 | Macadamia nut fatty acid | — | — | — | — | — | — | — | — | — | — |
| | phytosteryl *20 | | | | | | | | | | |
| 22 | Polyglyceryl-2 | — | — | — | — | — | — | — | — | — | — |
| | tetraisostearate | | | | | | | | | | |
| | (IOB: 0.17, viscosity: | | | | | | | | | | |
| | 369 mPa · s) *21 | | | | | | | | | | |
| 23 | Diisostearyl malate | 10 | 1.75 | 1.75 | 1.75 | 1.75 | 1.75 | 1.75 | — | 1.75 | 15 |
| | (IOB: 0.28, viscosity: | | | | | | | | | | |
| | 5500 mPa · s) *22 | | | | | | | | | | |
| 24 | Pentaerythrityl | — | — | — | — | — | — | — | — | — | — |
| | tetraethylhexanoate | | | | | | | | | | |
| | (IOB: 0.35, viscosity: | | | | | | | | | | |
| | 124 mPa · s) *23 | | | | | | | | | | |
| 25 | Triethylhexanoin | — | — | — | — | — | — | — | — | — | — |
| | (IOB: 0.36, viscosity: | | | | | | | | | | |
| | 30 mPa · s) *24 | | | | | | | | | | |
| 26 | Isononyl isononanoate | — | — | — | — | — | — | — | — | — | — |
| | (IOB: 0.20, viscosity: | | | | | | | | | | |
| | 7 mPa · s) | | | | | | | | | | |
| 27 | Diisopropyl adipate | — | — | — | — | — | — | — | — | — | — |
| | (IOB: 0.46, viscosity: | | | | | | | | | | |
| | 3 mPa · s) | | | | | | | | | | |
| 28 | Isostearyl neopentanoate | — | — | — | — | — | — | — | — | — | — |
| | (IOB: 0.14, viscosity: | | | | | | | | | | |
| | 10 mPa · s) | | | | | | | | | | |

TABLE 4-2
% by mass

| | | Example | | | | Comparative Example | | | | | |
|---|---|---|---|---|---|---|---|---|---|---|---|
| No. | Component | 30 | 31 | 32 | 33 | 1 | 2 | 3 | 4 | 5 | 6 |
| 29 | Polyglyceryl-2 isostearate | — | — | — | — | — | — | — | — | — | — |
| | (IOB: 0.81, viscosity: | | | | | | | | | | |
| | 6540 mPa · s) | | | | | | | | | | |
| 30 | Polyethylene wax *25 | — | 3 | — | — | — | — | — | — | — | — |
| 31 | Carnauba wax *26 | — | — | 3 | — | — | — | — | — | — | — |
| 32 | Microcrystalline wax *27 | — | — | — | 3 | — | — | — | — | — | — |
| 33 | Mineral oil | 6.8 | 6.8 | 6.8 | 6.8 | 6.8 | 6.8 | 6.8 | 6.8 | 6.8 | 6.8 |
| 34 | Dimethicone *28 | 2 | 2 | 2 | 2 | 2 | 2 | 2 | 2 | 2 | 2 |
| 35 | Talc | 45.8 | 51.05 | 51.05 | 51.05 | 61.95 | 72.05 | 60.55 | 55.8 | 45.55 | 40.8 |
| 36 | Iron oxide | 3 | 3 | 3 | 3 | 3 | 3 | 3 | 3 | 3 | 3 |

| | | | | | | | | | | |
|---|---|---|---|---|---|---|---|---|---|---|
| Content of (C) based on | 25 | 31 | 31 | 31 | 43 | 37 | 0 | 41 | 57 | 21 |
| total amount of non- | | | | | | | | | | |
| silicone oil agents (%) | | | | | | | | | | |
| Content of (D) based on | 38 | 8 | 8 | 8 | 12 | 10 | 16 | 0 | 7 | 48 |
| total amount of non- | | | | | | | | | | |
| silicone oil agents (%) | | | | | | | | | | |
| (A)/[(C) + (D)] | 0.31 | 0.63 | 0.63 | 0.63 | 0 | 0.63 | 2.95 | 0.80 | 0.31 | 0.24 |
| Content ratio of powders | 0.72 | 0.77 | 0.77 | 0.77 | 0.83 | 0.80 | 0.87 | 0.82 | 0.72 | 0.67 |
| based on total amount of | | | | | | | | | | |
| solid powder cosmetic | | | | | | | | | | |
| Content mass ratio of | 0.25 | 0.23 | 0.23 | 0.23 | 0.22 | 0 | 0.21 | 0.22 | 0.25 | 0.27 |
| (B) based on total | | | | | | | | | | |
| amount of powders | | | | | | | | | | |
| <Evaluation item/ | | | | | | | | | | |
| Results of determination> | | | | | | | | | | |
| Matte feeling | ◯ | ⊚ | ⊚ | ⊚ | X | X | Δ | ⊚ | ⊚ | X |
| Spreadability during application | ◯ | ◯ | ◯ | ⊚ | Δ | Δ | ◯ | Δ | X | Δ |
| Moist feeling | ⊚ | ⊚ | ⊚ | ⊚ | X | ◯ | X | X | ⊚ | ⊚ |
| Takeability of cosmetic | ⊚ | ◯ | ◯ | ◯ | ⊚ | ◯ | ⊚ | ⊚ | Δ | ◯ |
| Shock resistance | ⊚ | ⊚ | ⊚ | ⊚ | ◯ | ⊚ | X | ◯ | ⊚ | ⊚ |

TABLE 5-1
% by mass

| | | Comparative Example | | | | | | | | | |
|---|---|---|---|---|---|---|---|---|---|---|---|
| No. | Component | 7 | 8 | 9 | 10 | 11 | 12 | 13 | 14 | 15 | 16 |
| 1 | (Dimethicone/vinyl dimethicone) | — | — | — | — | — | — | — | — | — | — |
| | crosspolymer: 25%, | | | | | | | | | | |
| | dimethicone: 75% *1 | | | | | | | | | | |
| 2 | (Dimethicone/phenyl vinyl | — | — | — | — | — | — | — | — | — | — |
| | dimethicone) crosspolymer: | | | | | | | | | | |
| | 15%, diphenylsiloxy phenyl | | | | | | | | | | |
| | trimethicone: 85% *2 | | | | | | | | | | |
| 3 | (Vinyl dimethicone/lauryl | 3.9 | — | — | 3.9 | 3.9 | 3.9 | 3.9 | 3.9 | 3.9 | 3.9 |
| | dimethicone) crosspolymer: | | | | | | | | | | |
| | 30%, triethylhexanoin: 70% *3 | | | | | | | | | | |
| 4 | (PEG-15/lauryl dimethicone) | — | — | — | — | — | — | — | — | — | — |
| | crosspolymer: 30%, mineral | | | | | | | | | | |
| | oil: 70% *4 | | | | | | | | | | |
| 5 | (dimethicone/polyglycerol-3) | — | — | — | — | — | — | — | — | — | — |
| | crosspolymer: 25%, | | | | | | | | | | |
| | dimethicone: 75% *5 | | | | | | | | | | |
| 6 | Mixture of silica and | — | 7.9 | — | — | — | — | — | — | — | — |
| | dimethicone: solid content 30% | | | | | | | | | | |
| 7 | Dimethicone crosspolymer * 6 | 4 | — | — | 4 | 4 | 4 | 4 | 4 | 4 | 4 |
| 8 | (Vinyl dimethicone/ | — | — | — | — | — | — | — | — | — | — |
| | methicone silsesquioxane) | | | | | | | | | | |
| | crosspolymer *7 | | | | | | | | | | |
| 9 | Silica | — | — | 7.9 | — | — | — | — | — | — | — |
| 10 | Synthetic fluorophlogopite | 18 | 18 | 18 | — | 18 | 18 | 18 | 18 | 18 | 18 |
| | (aspect ratio: 35, average | | | | | | | | | | |
| | particle size: 12 μm) *9 | | | | | | | | | | |
| 11 | Synthetic fluorophlogopite | — | — | — | — | — | — | — | — | — | — |
| | (aspect ratio: 55, average | | | | | | | | | | |
| | particle size: 10 μm) *10 | | | | | | | | | | |
| 12 | Mica (aspect ratio: 70, | — | — | — | — | — | — | — | — | — | — |
| | average particle size: | | | | | | | | | | |
| | 10 μm) *11 | | | | | | | | | | |
| 13 | Boron nitride (aspect | — | — | — | — | — | — | — | — | — | — |
| | ratio: 31, average particle | | | | | | | | | | |
| | size: 15 pm) *12 | | | | | | | | | | |
| 14 | Synthetic fluorophlogopite | — | — | — | — | — | — | — | — | — | — |
| | (aspect ratio: 45, average | | | | | | | | | | |
| | particle size: 8 μm) *13 | | | | | | | | | | |
| 15 | Synthetic fluorophlogopite | — | — | — | — | — | — | — | — | — | — |
| | (aspect ratio: 75, average | | | | | | | | | | |
| | particle size: 20 μm) *14 | | | | | | | | | | |
| 16 | Synthetic fluorophlogopite | — | — | — | 18 | — | — | — | — | — | — |
| | (aspect ratio: 81, average | | | | | | | | | | |
| | particle size: 40 μm) *15 | | | | | | | | | | |
| 17 | Vaseline *16 | 6.5 | 6.5 | 6.5 | 6.5 | 6.5 | 6.5 | 6.5 | 6.5 | 1.5 | 6.5 |
| 18 | Dipentaerythrityl | — | — | — | — | — | — | — | — | — | — |
| | hexa (hydroxystearate/ | | | | | | | | | | |
| | stearate/rosinate) *17 | | | | | | | | | | |
| 19 | (Phytosteryl/isostearyl/ | — | — | — | — | — | — | — | — | — | — |
| | cetyl/stearyl/behenyl) | | | | | | | | | | |
| | dimer dilinoleate *18 | | | | | | | | | | |
| 20 | Di (octyldodecyl/ | — | — | — | — | — | — | — | — | — | — |
| | phytosteryl/behenyl) | | | | | | | | | | |
| | lauroyl glutamate *19 | | | | | | | | | | |
| 21 | Macadamia nut fatty | — | — | — | — | — | — | — | — | — | — |
| | acid phytosteryl *20 | | | | | | | | | | |
| 22 | Polyglyceryl-2 | — | — | — | — | — | — | — | — | — | — |
| | tetraisostearate | | | | | | | | | | |
| | (IOB: 0.17, viscosity: | | | | | | | | | | |
| | 369 mPa · s) *21 | | | | | | | | | | |
| 23 | Diisostearyl malate | 1.75 | 1.75 | 1.75 | 1.75 | — | — | — | — | 1.75 | 0.5 |
| | (IOB: 0.28, viscosity: | | | | | | | | | | |
| | 5500 mPa · s) *22 | | | | | | | | | | |
| 24 | Pentaerythrityl | — | — | — | — | — | — | — | — | — | — |
| | tetraethylhexanoate | | | | | | | | | | |
| | (IOB: 0.35, viscosity: | | | | | | | | | | |
| | 124 mPa · s) *23 | | | | | | | | | | |
| 25 | Triethylhexanoin | — | — | — | — | — | — | — | — | — | — |
| | (IOB: 0.36, viscosity: | | | | | | | | | | |
| | 30 mPa · s) *24 | | | | | | | | | | |
| 26 | Isononyl isononanoate | — | — | — | — | 1.75 | — | — | — | — | — |
| | (IOB: 0.20, viscosity: | | | | | | | | | | |
| | 7 mPa · s) | | | | | | | | | | |
| 27 | Diisopropyl adipate | — | — | — | — | — | 1.75 | — | — | — | — |
| | (IOB: 0.46, viscosity: | | | | | | | | | | |
| | 3 mPa · s) | | | | | | | | | | |
| 28 | Isostearyl neopentanoate | — | — | — | — | — | — | 1.75 | — | — | — |
| | (IOB: 0.14, viscosity: | | | | | | | | | | |
| | 10 mPa · s) | | | | | | | | | | |

TABLE 5-2
% by mass

| | | Comparative Example | | | | | | | | | |
|---|---|---|---|---|---|---|---|---|---|---|---|
| No. | Component | 7 | 8 | 9 | 10 | 11 | 12 | 13 | 14 | 15 | 16 |
| 29 | Polyglyceryl-2 isostearate | — | — | — | — | — | — | — | 1.75 | — | — |
| | (IOB: 0.81, viscosity: | | | | | | | | | | |
| | 6540 mPa · s) | | | | | | | | | | |
| 30 | Polyethylene wax *25 | — | — | — | — | — | — | — | — | — | — |
| 31 | Carnauba wax *26 | — | — | — | — | — | — | — | — | — | — |
| 32 | Microcrystalline wax *27 | — | — | — | — | — | — | — | — | — | — |
| 33 | Mineral oil | 6.8 | 6.8 | 6.8 | 6.8 | 6.8 | 6.8 | 6.8 | 6.8 | 6.8 | 6.8 |
| 34 | Dimethicone *28 | 2 | 2 | 2 | 2 | 2 | 2 | 2 | 2 | 2 | 2 |
| 35 | Talc | 49.05 | 54.05 | 54.05 | 54.05 | 54.05 | 54.05 | 54.05 | 54.05 | 54.05 | 54.05 |
| 36 | Iron oxide | 3 | 3 | 3 | 3 | 3 | 3 | 3 | 3 | 3 | 3 |

| | | | | | | | | | | |
|---|---|---|---|---|---|---|---|---|---|---|
| Content of (C) based on | 29 | 43 | 43 | 37 | 37 | 37 | 37 | 37 | 12 | 39 |
| total amount of non- | | | | | | | | | | |
| silicone oil agents (%) | | | | | | | | | | |
| Content of (D) based on | 8 | 12 | 12 | 10 | 0 | 0 | 0 | 0 | 14 | 3 |
| total amount of non- | | | | | | | | | | |
| silicone oil agents (%) | | | | | | | | | | |
| (A)/[(C) + (D)] | 0.63 | 0 | 0 | 0.63 | 0.80 | 0.80 | 0.80 | 0.80 | 1.59 | 0.74 |
| Content ratio of powders | 0.75 | 0.75 | 0.83 | 0.80 | 0.80 | 0.80 | 0.80 | 0.80 | 0.80 | 0.80 |
| based on total amount of | | | | | | | | | | |
| solid powder cosmetic | | | | | | | | | | |
| Content mass ratio of | 0.24 | 0.24 | 0.22 | 0 | 0.22 | 0.22 | 0.22 | 0.22 | 0.22 | 0.22 |
| (B) based on total | | | | | | | | | | |
| amount of powders | | | | | | | | | | |
| <Evaluation item/ | | | | | | | | | | |
| Results of determination> | | | | | | | | | | |
| Matte feeling | ⊚ | X | X | X | ⊚ | ⊚ | ⊚ | ⊚ | Δ | ⊚ |
| Spreadability during application | Δ | Δ | Δ | ◯ | X | X | ◯ | X | ◯ | ◯ |
| Moist feeling | ◯ | X | X | Δ | ◯ | ◯ | X | ⊚ | X | X |
| Takeability of cosmetic | X | ⊚ | ⊚ | ⊚ | ⊚ | ⊚ | ◯ | Δ | ⊚ | ⊚ |
| Shock resistance | ⊚ | ◯ | ◯ | Δ | Δ | Δ | ◯ | ⊚ | Δ | ◯ |

1 KSG-16 (manufactured by Shin-Etsu Chemical Co., Ltd.)
2 KSG-18A (manufactured by Shin-Etsu Chemical Co., Ltd.)
3 KSG-43 (manufactured by Shin-Etsu Chemical Co., Ltd.)
4 KSG-310 (manufactured by Shin-Etsu Chemical Co., Ltd.)
5 KSG-710 (manufactured by Shin-Etsu Chemical Co., Ltd.)
6 DOWSIL EP-9610 COSMETIC POWDER (manufactured by Dow Toray Co., Ltd.)
7 KSP-100 (manufactured by Shin-Etsu Chemical Co., Ltd.)
9 NK-M (manufactured by NIHON KOKEN KOGYO CO., LTD.)
10 NK-10G (manufactured by NIHON KOKEN KOGYO CO., LTD.)
11 Y-1800 (manufactured by YAMAGUCHI MICA CO., LTD.)
12 CCS102-JA BORON NITRIDE POWDER (manufactured by Momentive Performance Materials Inc.)
13 NK-8G (manufactured by NIHON KOKEN KOGYO CO., LTD.)
14 NK-20G (manufactured by NIHON KOKEN KOGYO CO., LTD.)
15 PDM-40L (manufactured by TOPY INDUSTRIES, LIMITED)
16 SNOW WHITE SPECIAL (manufactured by Sonneborn LLC)
17 COSMOL 168ARV (manufactured by The Nisshin Oillio Group, Ltd.)
18 PLANDOOL-S (manufactured by NIPPON FINE CHEMICAL CO., LTD.)
19 PLANDOOL-LG1 (manufactured by NIPPON FINE CHEMICAL CO., LTD.)
20 PLANDOOL-MAS (manufactured by NIPPON FINE CHEMICAL CO., LTD.)
21 COSMOL 44V (manufactured by The Nisshin Oillio Group, Ltd.)
22 COSMOL 222 (manufactured by The Nisshin Oillio Group, Ltd.)
23 SALACOS 5408 (manufactured by The Nisshin Oillio Group, Ltd.)
24 T. I. O (manufactured by The Nisshin Oillio Group, Ltd.)
25 Synthetic wax P-200 (manufactured by NATURAL-PRODUCTS co., ltd.)
26 Carnauba wax (manufactured by Machard Chemical)
27 MULTIWAX W445 (manufactured by Sonneborn LLC)
28 Silicon KF-96A (6CS) (manufactured by Shin-Etsu Chemical Co., Ltd.).

In the above examples, the components (1) to (5) and (7) to (8) are classified into the component (A). The components (10) to (15) are classified into the component (B). The components (17) to (21) are classified into the component (C). The components (22) to (25) are classified into the component (D). The components (30) to (32) are waxes. The oil agents in the components (3) and (4) and the components (17) to (33) are non-silicone oils.

### (Production Method: Tables 1 to 5)

A. The components (6) to (16) and (34) to (36) were uniformly mixed.

B. The components (1) to (5) and (17) to (33) were warmed to 70° C., uniformly mixed, and cooled to room temperature.

C. B was added to A to prepare cosmetic bases. To 100 parts by mass of each cosmetic base was added 20 parts by mass of isododecane. The mixtures were kneaded. Resin containers were filled with the mixtures. The isododecane was removed while the mixture was compressed. The resultants were then dried at 70° C. for 8 hours to obtain the solid powder cosmetics (eye shadows).

### (Evaluation Method)

The cosmetics were evaluated with respect to the following evaluation items by the following methods.

### (Evaluation Items)

a. Matte feel
b. Spreadability during application
c. Moist feeling
d. Takeability of cosmetic
e. Shock resistance

Ten special panelists for evaluating cosmetics used and tested each sample with respect to the evaluation items a to d, and evaluated the sample on a scale of zero to six to give scores to the sample based on the following absolute evaluation criteria. The total scores of the sample given by all the panelists were used to calculate the average values thereof, followed by determination based on the following four-grade criteria.

The following evaluation items were evaluated by the following methods.

### (Evaluation Items): (Evaluation Viewpoints)

Evaluation item a: Each sample was applied to eyelids, and it was visually evaluated whether the matte feeling was perceived.
Evaluation item b: Each sample was applied to eyelids. It was visually evaluated whether the preparation can be uniformly applied.
Evaluation item c: Each sample was applied to eyelids. It was evaluated whether the dry feeling was perceived with the sample applied.
Evaluation item d: If each sample was taken with the fingers, it was evaluated whether favorable takeability was felt.

### (Absolute Evaluation Criteria)

### (Score): (Evaluation)

6: Very good
5: Good
4: Slightly good
3: Fair
2: Slightly poor
1: Poor
0: Very poor

### (Four-Grade Criteria)

### (Determination): (Average Scores)

⊙: More than 5 points: Very good
∘: More than 3.5 points and 5 points or less: Good
Δ: More than 2 points and 3.5 points or less: Slightly poor
x: 2 points or less: Poor

To examine the evaluation item e, five samples as each of the solid powder cosmetics of Examples and Comparative Examples were dropped from a height of 50 cm to a wooden hard plate. It was visually observed whether the dropped samples were fractured, cracked, fissured, or exfoliated. The occurrence level thereof was evaluated on a five-grade scale as the evaluation item e.

### <Criteria>

### (Results of Evaluation): (Determination)

None of the five are changed: ⊙
One of the five is slightly changed: ∘
Two of the five are changed: Δ
Three or more of the five are changed: x

As is clear from the results in Tables 1 to 5, Examples 1 to 33 were solid powder cosmetics that are excellent in all the evaluation items.

Meanwhile, Comparative Example 1, not containing the component (A), exhibited a strong dry feeling, and was not satisfactory in the matte feeling, either.

Comparative Example 2, not containing the component (B), was not satisfactory in spreadability during application and the matte feeling. Comparative Example 3, not containing the component (C), was inferior in shock resistance, was likely to crack or fracture when the sample was dropped, and was not satisfactory in the matte feeling or the moist feeling, either. Comparative Example 4, not containing the component (D), was likely to feel dry, and was poor in spreadability, either. Comparative Example 5, containing the component (C) at a content of more than 50% based on the total amount of non-silicone oils, was poor in takeability of the cosmetic, and was not satisfactory in spreadability during application, either. Comparative Example 6, containing the component (D) at a content of more than 45% based on the total amount of non-silicone oils, was inferior in the matte feeling, and was not satisfactory in spreadability. Comparative Example 7, containing the component (E) at a content of more than 3%, was difficult to take, and was not satisfactory in spreadability, either. Comparative Example 8, containing a mixture of silica and dimethicone instead of the component (A), felt dry, and was not satisfactory in the matte feeling, either. Comparative Example 9, containing silica instead of the component (A), was not satisfactory in the moist feeling or the matte feeling, either. Comparative Example 10, containing synthetic fluorophlogopite having an average particle size of more than 20 μm instead of the component (B), was inferior in shock resistance, and was not consequently satisfactory in the matte feeling, either. Comparative Examples 11 and 12, containing ester oils having viscosities of less than 10 mPa·s instead of the component (D), were not consequently satisfactory in spreadability during application. Comparative Example 13, containing an ester oil having an IOB value of less than 0.15 instead of the component (D), was likely to feel dry. Comparative Example 14, containing an ester oil agent having an IOB value of more than 0.50 instead of the component (D), was inferior in spreadability during application. Comparative Example 15, containing the component (C) at a content of less than 15% based on the total amount of the non-silicone oil agents, felt dry, and was not satisfactory in a matte feeling or shock resistance, either. Comparative Example 16, containing the component (D) at a content of less than 5% based on the total amount of the non-silicone oil agents, was markedly inferior in the moist feeling.

### Example 34: Solid Powder Cosmetic (Foundation)

A solid powder cosmetic (foundation) was produced in accordance with the following prescription by the following method.

| (Prescription) | (%) |
|---|---|
| (1) 2% Triethoxycaprylylsilane-treated titanium oxide | 15 |
| (2) Blood red | 1 |
| (3) Yellow iron oxide | 3 |
| (4) Black iron oxide | 0.5 |
| (5) (Vinyl dimethicone/methicone silsesquioxane) | 5 |
| crosspolymer *7 (component A) | |
| (6) Synthetic fluorophlogopite *9 (component B) | 10 |
| (7) Talc | Remainder |
| (8) Silicic anhydride | 5 |
| (9) N-Lauroyl-L-lysine | 5 |
| (10) Barium sulfate | 5 |
| (11) Zinc stearate *29 | 5 |
| (12) Swollen (Dimethicone/phenyl vinyl dimethicone) | 10 |
| crosspolymer · dimethicone (solid content: 15%) *2 | |
| (component A) | |
| (13) Dextrin (Palmate/2-ethylhexanoate) *30 | 1.5 |
| (14) Vaseline *16 (component C) | 2 |
| (15) Diisostearyl malate *22 (component D) | 2 |
| (16) Mineral oil | 2 |

*29 NISSAN ELECTOL MZ-2 (manufactured by NOF CORPORATION)
*30 Rheopearl TT2 (manufactured by Chiba Flour Milling Co., Ltd.)

### (Production Method)

A. The components (1) to (11) were uniformly mixed.
B. The components (13) to (16) were heated and dissolved, and then cooled to room temperature to be gelated.
C. To B was added the component (12), followed by mixing.
D. C was added to A, followed by mixing. The mixture was pulverized.
E. A metal tray is filled with D to obtain the solid powder cosmetic (foundation).

The non-silicone oil agents were the components (14) to (16). The content of the component (C) based on the total content of non-silicone oil agents is 33% by mass. The content of the component (D) based on the total content of non-silicone oil agents is 33% by mass. (A)/[(C)+(D)]=1.62, the content ratio of the powders to the total amount of the solid powder cosmetic was 0.84, and the content mass ratio of the component (B) to the total amount of the powders was 0.12. The obtained solid powder cosmetic (foundation) was good in all the items, namely the “matte feeling”, “spreadability during application”, the “moist feeling”, “takeability of the cosmetic”, and “shock resistance”.

### Example 35 Solid Powder Cosmetic (Cheek Shadow)

A solid powder cosmetic (cheek shadow) was produced in accordance with the following prescription by the following method.

| (Prescription) | (%) |
|---|---|
| (1) 2% Silicone-treated talc | 20 |
| (2) Synthetic fluorophlogopite *9 (component B) | 10 |
| (3) Boron nitride *12 (component B) | 10 |
| (4) Talc | Remainder |
| (5) Zinc oxide | 2 |
| (6) Dimethicone crosspolymer *6 (component A) | 5 |
| (7) Red No. 226 | 0.5 |
| (8) Yellow No. 4 | 1 |
| (9) Calcium stearate *31 | 10 |
| (10) Swollen (vinyl dimethicone/lauryl dimethicone) | 4 |
| crosspolymer · triethylhexanoin (solid content: 30%) *3 | |
| (component A) | |
| (11) Swollen (dimethicone/polyglycerol-3) | 2 |
| crosspolymer · dimethicone | |
| (solid content: 25%) *5 (component A) | |
| (12) Dextrin palmitate *32 | 1 |
| (13) Triethylhexanoin *24 (component D) | 2 |
| (14) Vaseline *16 (component C) | 3 |
| (15) Diisostearyl malate *22 (component D) | 1 |
| (16) Mineral oil | 4 |

*31 NISSAN ELECTOL MC-2 (manufactured by NOF CORPORATION)
*32 Rheopearl KL2 (manufactured by Chiba Flour Milling Co., Ltd.)

### (Production Method)

A. The components (1) to (9) were uniformly mixed.
B. The components (11) to (16) were heated and dissolved, and then cooled to room temperature to be gelated.
C. To B was added the component (10), followed by mixing.
D. C was added to A, followed by mixing. The mixture was pulverized.
E. A metal tray was filled with D to obtain the solid powder cosmetic (cheek shadow).

The non-silicone oil agents were the component (10) and the components (12) to (16). The content of the component (C) based on the total content of non-silicone oil agents is 23% by mass. The content of the component (D) based on the total content of non-silicone oil agents is 23% by mass. (A)/[(C)+(D)]=0.28, the content ratio of the powders to the total amount of the solid powder cosmetic was 0.85, and the content mass ratio of the component (B) to the total amount of the powders was 0.12.

The obtained solid powder cosmetic (cheek shadow) was good in all the items, namely the “matte feeling”, “spreadability during application”, the “moist feeling”, “takeability of the cosmetic”, and “shock resistance”.

The present application is based on Japanese Patent Application No. 2022-166537, filed on Oct. 17, 2022, the disclosed contents of which are incorporated herein by reference in their entirety.

What is claimed is:

1. A solid powder cosmetic comprising components (A) to (D):
(A) a crosslinked organopolysiloxane polymer comprising an alkyl branching chain and/or a phenyl branching chain;
(B) one or more plate-like powders selected from the group consisting of: mica having an average particle size of 5 to 20 μm, synthetic fluorophlogopite having an average particle size of 5 to 20 μm, and boron nitride having an average particle size of 5 to 20 μm;
(C) an oil agent that is pasty at 25° C.; and
(D) an ester oil having an IOB value of 0.15 to 0.50 and a viscosity of 10 to 10000 mPa's at 20° C.,
wherein a content of the component (C) is 15 to 50% by mass based on a total amount of non-silicone oil agents,
a content of the component (D) is 5 to 45% by mass based on the total amount of the non-silicone oil agents, and
the cosmetic is free of a wax, or a content of the wax is 3% by mass or less based on a total amount of the cosmetic.

2. (canceled)

3. The solid powder cosmetic according to claim 1, wherein an aspect ratio of the component (B) is 30 to 75.

4. The solid powder cosmetic according to claim 1, wherein a content mass ratio of the component (B) to a total amount of the powders, (B)/(total amount of the powders), is 0.05 to 0.40.

5. The solid powder cosmetic according to claim 1, wherein a content mass ratio of the powders to the total amount of the solid powder cosmetic is 0.60 to 0.85.

6. The solid powder cosmetic according to claim 1, wherein the component (C) is one or more selected from the group consisting of hydrocarbon oils, dimer acid esters, sterol fatty acid esters, and dipentaerythritol fatty acid esters.

7. The solid powder cosmetic according to claim 1, wherein the IOB value of the component (D) is 0.15 to 0.35.

8. The solid powder cosmetic according to claim 1, wherein a content mass ratio of the component (A) to a total mass of the components (C) and (D), (A)/[(C)+(D)], is 0.25 to 2.20.

9. The solid powder cosmetic according to claim 8, wherein an aspect ratio of the component (B) is 30 to 75.

10. The solid powder cosmetic according to claim 8, wherein a content mass ratio of the component (B) to a total amount of the powders, (B)/(total amount of the powders), is 0.05 to 0.40.

11. The solid powder cosmetic according to claim 8, wherein a content mass ratio of the powders to the total amount of the solid powder cosmetic is 0.60 to 0.85.

12. The solid powder cosmetic according to claim 8, wherein the component (C) is one or more selected from the group consisting of hydrocarbon oils, dimer acid esters, sterol fatty acid esters, and dipentaerythritol fatty acid esters.

13. The solid powder cosmetic according to claim 8, wherein the IOB value of the component (D) is 0.15 to 0.35.
